(19) United States
(12) Patent Application Publication
(10) Pub. No.: US 2007/0140408 A1
(43) Pub. Date: Jun. 21, 2007

(54) X-RAY CT IMAGING METHOD AND X-RAY CT APPARATUS

(76) Inventors: Yasuro Takiura, Tokyo (JP); Akihiko Nishide, Tokyo (JP); Takashi Fujishige, Tokyo (JP)

Correspondence Address: Patrick Rasche;Armstrong Teasdale, LLP, One Metropolitan Square, Suite 2600, St. Louis, MO, 63102, US

(21) Appl. No.: 11/564,628

(22) Filed: Nov. 29, 2006

(30) Foreign Application Priority Data
Dec. 5, 2005 (JP) 2005-350700

(51) Int. Cl. H05G 1/60; A61B 6/00; G01N 23/00; G21K 1/12

(52) U.S. Cl. 378/4

(57) ABSTRACT

Enhancement of the resolution of tomograms obtained by conventional scanning (axial scanning), cine-scanning, helical scanning or variable-pitch helical scanning by the X-ray CT apparatus using a multi-row X-ray detector or a two-dimensional X-ray area detector of a matrix structure is to be realized by a simple method. An X-ray CT apparatus is realized in which a multi-row X-ray detector or a two-dimensional X-ray area detector of a matrix structure with a small amount of processing work, and image reconstructing device capable of providing high-resolution tomograms by image reconstruction is provided.

### BACKGROUND OF THE INVENTION

The present invention relates to an X-ray CT apparatus for medical use or an X-ray CT apparatus for industrial use, an X-ray CT (Computed Tomography) imaging method and an X-ray CT apparatus, and to enhancing the resolution of tomograms simply fabricated X-ray detectors of conventional scanning (axial scanning), cine-scanning, helical scanning or variable-pitch helical scanning.

Conventionally, in a multi-row X-ray detector-based X-ray CT apparatus or an X-ray CT apparatus using a two-dimensional X-ray area detector of a matrix structure, a multi-row X-ray detector or a two-dimensional X-ray area detector of a square lattice or rectangular lattice structure as shown in FIG. 15 was used as described in JP-A No. 193750/2000. In this case, wherein the resolution of the X-ray detector was to be enhanced, the width of each had to be reduced to 1/n (where n is an integer) both in the channel direction and the row direction as shown in FIG. 16, but this was a problem from the viewpoint of the manufacturing difficulty of the X-ray detector.

Thus for the conventional multi-row X-ray detector or two-dimensional X-ray area detector, a circular type multi-row X-ray detector of FIG. 18(a), a planar type two-dimensional X-ray area detector of FIG. 18(b) or a two-dimensional X-ray area detector combining a plurality of planar type X-ray detectors of FIG. 18(c) was fabricated by combining X-ray detector modules of a square lattice structure as shown in FIGS. 18, and used in an X-ray CT apparatus.

This also poses a problem from the viewpoint that the volume rate of the reflectors within the X-ray detector module increases, resulting in a drop in the efficiency of X-ray acquisition and accordingly in a performance deterioration of the X-ray detector.

As one example of way to fabricate the X-ray detector module in this case, as shown in FIGS. 19, first a plate type scintillator was cut in the channel direction, reflectors were placed on the cut sections, which were joined again; next, it was cut in the row direction, reflectors were placed and the cut pieces were joined to produce a detector module of a matrix structure of square lattice or rectangular lattice. However, as the requirement for higher resolution of X-ray detectors became more stringing, if it is attempted to achieve a resolution twice as fine in the channel direction and twice as fine in the row direction, division of the X-ray detector or X-ray detector module of FIG. 15 into each channel direction or each row direction as is the case with the X-ray detector or X-ray detector module of FIG. 16 was needed, which was a problem from the viewpoint of the manufacturing difficulty of the X-ray detector or X-ray detector module.

However, in a multi-row X-ray detector-based X-ray CT apparatus or an X-ray CT apparatus using a two-dimensional X-ray area detector, the requirement for higher resolution of X-ray detectors is expected to become more stringent in the future.

### SUMMARY OF THE INVENTION

Therefore, an object of the present invention is to make it possible to realize by a simple method achievement of higher X-ray detector resolution for multi-row X-ray detectors or two-dimensional X-ray area detectors of a matrix structure, and to realize enhancement of the resolution of tomograms by an X-ray CT apparatus using such X-ray detectors by conventional scanning (axial scanning), cine-scanning, helical scanning or variable-pitch helical scanning.

The present invention solves the problems noted above by providing an X-ray CT apparatus or an X-ray CT imaging method characterized in that it realizes an X-ray CT apparatus in which a multi-row X-ray detector or a two-dimensional X-ray area detector of a matrix structure constructs a high-resolution multi-row X-ray detector with a small amount of processing work, and in which image reconstructing device is provided being capable of providing high-resolution tomograms by image reconstruction.

According to a first aspect of the invention, there is provided an X-ray CT apparatus comprising: X-ray data acquisition device for acquiring projection data of an X-ray passed through a subject positioned between an X-ray generator and an X-ray detector which are opposite to each other; image reconstructing device for performing image reconstruction from the projection data acquired from that X-ray data acquisition device; image display device for displaying a tomographic image obtained by said image reconstructing device; and imaging condition setting device for setting various image acquisition parameters for acquisition of a tomographic image, wherein said X-ray detector includes a detector of which the X-ray detector module is divided into X-ray detector channels by parallel lines in three or more directions.

According to a second aspect of the invention, there is provided an X-ray CT apparatus according to the first aspect wherein said X-ray detector includes a multi-row X-ray detector.

According to a third aspect of the invention, there is provided an X-ray CT apparatus according to the first aspect wherein said X-ray detector includes a two-dimensional X-ray area detector.

In the X-ray CT apparatus according to the first aspect to third aspect, since the X-ray detector module is divided into X-ray detector channels by parallel lines in three or more directions, the structure is easy to fabricate.

According to a fourth aspect of the invention, there is provided an X-ray CT apparatus according to the first aspect characterized in that it has X-ray data acquisition device of which each X-ray detector channel has a triangular shape.

The X-ray CT apparatus according to the fifth aspect, since each X-ray detector channel has a triangular shape, the structure is easy to fabricate.

According to a fifth aspect of the invention, there is provided an X-ray CT apparatus comprising: X-ray data acquisition device for acquiring projection data of an X-ray passed through a subject positioned between an X-ray generator and an X-ray detector which are opposite to each other; image reconstructing device for performing image reconstruction from the projection data acquired from that X-ray data acquisition device; image display device for displaying a tomographic image obtained by said image reconstructing device; and imaging condition setting device for setting various image acquisition parameters for acquisition of a tomographic image, wherein said image reconstructing device includes three-point weighted addition processing or three-point interpolation processing.

The X-ray CT apparatus according to the fifth aspect, since data which are three-dimensionally back-projected or two-dimensionally back-projected to certain pixels in a tomogram from X-ray projection data are extracted by using three-point weighted addition processing or three-point interpolation processing, the X-ray projection data can be three-dimensionally back-projected or two-dimensionally back-projected without being blurred and tomograms can be obtained without deterioration of their spatial resolution.

According to a sixth aspect of the invention, there is provided an X-ray CT apparatus according to the first aspect characterized in that it has image reconstructing device which uses three-point weighted addition processing or three-point interpolation processing.

The X-ray CT apparatus according to the sixth aspect, since data which are three-dimensionally back-projected or two-dimensionally back-projected to certain pixels in a tomogram from X-ray projection data are extracted by using three-point weighted addition processing or three-point interpolation processing, the X-ray projection data can be three-dimensionally back-projected or two-dimensionally back-projected without being blurred and tomograms can be obtained without deterioration of their spatial resolution.

According to a seventh aspect of the invention, there is provided an X-ray CT apparatus according to the first aspect characterized in that it has image reconstructing device which uses four-point weighted addition processing or four-point interpolation processing.

The X-ray CT apparatus according to the seventh aspect, since data which are three-dimensionally back-projected or two-dimensionally back-projected to certain pixels in a tomogram from X-ray projection data are extracted by using four-point weighted addition processing or four-point interpolation processing, weighted addition coefficients or interpolation coefficients can be easily figured out.

According to a eighth aspect of the invention, there is provided an X-ray CT apparatus according to the first aspect characterized in that it has image reconstructing device which uses two-point weighted addition processing or two-point interpolation processing.

The X-ray CT apparatus according to the eighth aspect, since data which are three-dimensionally back-projected or two-dimensionally back-projected to certain pixels in a tomogram from X-ray projection data are extracted by using two-point weighted addition processing or two-point interpolation processing, weighted addition coefficients or interpolation coefficients can be easily figured out.

According to an ninth aspect of the invention, there is provided an X-ray CT apparatus according to the first aspect characterized in that it has image reconstructing device which uses nearest neighbor processing.

The X-ray CT apparatus according to the ninth aspect, since data which are three-dimensionally back-projected or two-dimensionally back-projected to certain pixels in a tomogram from X-ray projection data are extracted by using nearest neighbor processing, weighted addition coefficients or interpolation coefficients can be easily figured out.

According to a 10th aspect of the invention, there is provided an X-ray CT apparatus according to the first aspect characterized in that it has image reconstructing device which uses three-dimensional image reconstruction processing.

The X-ray CT apparatus according to the 10th aspect, since it performs image reconstruction by using three-dimensional image reconstruction processing, can give a tomogram of high picture quality little affected by artifact whether at the center of the tomogram or in a position away from the center of image reconstruction. Moreover, whether by conventional scanning (axial scanning) or cine-scanning or if the tomogram is on an outer X-ray detector row away in the z direction, a tomogram of high picture quality little affected by artifact can be obtained.

According to a 11th aspect of the invention, there is provided an X-ray CT apparatus according to the ninth aspect characterized in that it has image reconstructing device which, when conventional scanning (axial scanning) or cine-scanning is performed, can achieve image reconstruction of a tomogram of any desired slice thickness in any z-direction coordinate position.

The X-ray CT apparatus according to the 11th aspect, since it performs image reconstruction by using three-dimensional image reconstruction processing, can achieve image reconstruction of a tomogram of any desired slice thickness in any z-direction coordinate position in conventional scanning (axial scanning) or cine-scanning.

According to an 12th aspect of the invention, there is provided an X-ray CT apparatus according to the ninth aspect characterized in that it has image reconstructing device which, when helical scanning or variable-pitch helical scanning is performed, can achieve image reconstruction of a tomogram of any desired slice thickness in any z-direction coordinate position.

The X-ray CT apparatus according to the 12th aspect, since it performs image reconstruction by using three-dimensional image reconstruction processing, can achieve image reconstruction of a tomogram of any desired slice thickness in any z-direction coordinate position in helical scanning or variable-pitch helical scanning.

According to a 13th aspect of the invention, there is provided an X-ray CT apparatus according to 11th aspect characterized in that it has image reconstructing device which alternately rearranges and interleaves X-ray projection data on adjoining rows, reconstructs high-resolution X-ray projection data and performs image reconstruction of the X-ray projection data.

According to a 14th aspect of the invention, there is provided an X-ray CT apparatus according to the 12th aspect characterized in that it has image reconstructing device which alternately rearranges and interleaves X-ray projection data on adjoining rows, reconstructs high-resolution X-ray projection data and performs image reconstruction of the X-ray projection data.

The X-ray CT apparatus according to the 13th or 14 th aspect can enhance the resolution of X-ray detector data in the channel direction by alternately inserting and interleaving X-ray detector data on adjoining rows, and accordingly can improve the spatial resolution of tomograms.

According to a 15th aspect of the invention, there is provided an X-ray CT apparatus according to the 12th aspect characterized in that it has image reconstructing device which alternately rearranges and interleaves X-ray projection data on adjoining rows in the case of a high-frequency reconstruction function.

According to a 16th aspect of the invention, there is provided an X-ray CT apparatus according to the 12th aspect characterized in that it has image reconstructing device which alternately rearranges and interleaves X-ray projection data on adjoining rows in the case of a high-frequency reconstruction function.

The X-ray CT apparatus according to the 15th or 16 th aspect can enhance the resolution of X-ray detector data in the channel direction by alternately inserting and interleaving X-ray detector data on adjoining rows especially when image reconstruction is performed with a high-frequency reconstruction function, and accordingly can improve the spatial resolution of tomograms.

According to a 17th aspect of the invention, there is provided an X-ray CT apparatus comprising X-ray data acquisition device which, while rotating an X-ray generating device and a multi-row X-ray detector which detects X-rays in an opposing manner or a two-dimensional X-ray area detector of a matrix structure around a rotation center position in-between, collects X-ray projection data transmitted by a subject positioned in-between; image reconstructing device which performs image reconstruction from the projection data collected from that X-ray data acquisition device; image display device which displays a tomogram having undergone image reconstruction; and imaging condition setting device which sets various imaging conditions of tomography, the X-ray CT apparatus being characterized in that it has image reconstructing device which uses three-point weighted addition processing or three-point interpolation processing in weighted addition processing or interpolation processing in image reconstruction.

The X-ray CT apparatus according to the 17th aspect, since it uses three-point weighted addition processing or three-point interpolation processing, can perform image reconstruction with minimized blurring of X-ray projection data and obtain high-resolution tomograms.

The X-ray CT apparatus or the X-ray CT image reconstructing method according to the invention can realize a high resolution for a multi-row X-ray detector or a two-dimensional X-ray area detector of a matrix structure by a simple method, and provides the effect of achieving a high resolution for tomograms by conventional scanning (axial scanning), cine-scanning, helical scanning or variable-pitch helical scanning by the X-ray CT apparatus using such X-ray detectors.

### BRIEF DESCRIPTION OF THE DRAWINGS

FIG. 1 is a block diagram of an X-ray CT apparatus in one mode for carrying out the present invention.

FIG. 2 is a diagram illustrating an X-ray generating device (X-ray tube) and a multi-row X-ray detector as viewed on the xy plane.

FIG. 3 is a diagram illustrating an X-ray generating device (X-ray tube) and a multi-row X-ray detector as viewed on the yz plane.

FIG. 4 is a flow chart showing the flow of imaging a subject.

FIG. 5 is a flow chart outlining the operation of the X-ray CT apparatus pertaining to one embodiment of the invention.

FIG. 6 is a flow chart showing details of pre-treatments.

FIG. 7 is a flow chart showing details of three-dimensional image reconstruction processing.

FIGS. 8(a), 8(b) are conceptual diagrams showing a state of projecting lines on a reconstruction area in the X-ray transmitting direction.

FIG. 9 is a conceptual diagram showing lines projected on detector faces.

FIG. 10 is a conceptual diagram showing a state of projecting projection data Dr(view, x, y) on the reconstruction area.

FIG. 11 is a conceptual diagram showing back-projection pixel data D2 of pixels on the reconstruction area.

FIG. 12 is a diagram illustrating a state in which back-projection data D3 are obtained by subjecting the back-projection pixel data D2 to all-view addition pixel by pixel.

FIG. 13 is a conceptual diagram showing a state of projecting lines on a circular reconstruction area in the X-ray transmitting direction.

FIG. 14 is a diagram showing an imaging condition input screen for the X-ray CT apparatus.

FIG. 15 is a diagram showing a conventional system.

FIG. 16 is a diagram showing achievement of higher resolution by a conventional method.

FIG. 17 is a diagram showing a method proposed herein.

FIG. 18(a) is a diagram showing a circular type multi-row X-ray detector.

FIG. 18(b) is a diagram showing a planar type two-dimensional X-ray area detector.

FIG. 18(c) is a diagram showing a two-dimensional X-ray area detector combining a plurality of planar type X-ray detectors.

FIG. 19 is a diagram showing a way of fabricating a conventional X-ray detector module.

FIG. 20 is a diagram showing a way of fabricating an X-ray detector module of this embodiment.

FIG. 21 is a diagram showing an eight-channel eight-row X-ray detector module.

FIG. 22 is a diagram showing a 16-channel 16-row X-ray detector module.

FIG. 23 is a diagram showing Example 1 of 16-channel 16-row X-ray detector module of this embodiment.

FIG. 24 is a diagram showing a 32-channel 16-row X-ray detector module.

FIG. 25 is a diagram showing back projection processing by four-point weighted addition.

FIG. 26 is a diagram showing back projection processing by four-point interpolation.

FIG. 27 is a diagram showing projection data arrayed in a hound's tooth check pattern.

FIG. 28 is a diagram showing hound's tooth check four-point weighted addition.

FIG. 29 is a diagram showing square lattice four-point weighted addition.

FIG. 30 is a diagram showing hound's tooth check three-point weighted addition.

FIG. 31 is a diagram showing square lattice three-point weighted addition.

FIG. 32 is a diagram showing a data extracting method by weighted addition using three points.

FIG. 33 is a diagram showing comparison of a data extracting method by weighted addition using three points and a data extracting method by weighted addition using four points.

FIG. 34 is a diagram showing a lattice coordinate system (Cartesian system).

FIG. 35 is a diagram showing lattice coordinates of an image-reconstructed tomogram and a locus line of back projection processing.

FIG. 36 is a diagram showing Example 2 of detector module of this embodiment.

FIG. 37 is a diagram showing Example 1 of adjoining X-ray detector modules.

FIG. 38 is a diagram showing Example 2 of adjoining X-ray detector modules.

FIG. 39 is a diagram showing a 16-channel 16-row X-ray detector module.

FIG. 40 is a diagram showing a 32-channel 16-row X-ray detector module.

FIG. 41 is a diagram showing Example 1 of 16-channel 16-row X-ray detector module of this embodiment.

FIG. 42 is a diagram showing Example 2 of 16-channel 16-row X-ray detector module of this embodiment.

FIG. 43 is a diagram showing treatment of projection data of mutually close rows as interleaved one-dimensionally arrayed data.

FIG. 44 is a diagram showing a rectangular X-ray detector module.

FIG. 45 is a diagram showing a parallelogrammatic X-ray detector module.

FIG. 46 is a diagram showing an outline of selecting three points for three-point interpolation in Embodiment 1.

FIG. 47 is a diagram showing details of selecting three points for three-point interpolation in Embodiment 1.

FIG. 48 is a diagram showing an outline of selecting three points for three-point interpolation in Embodiment 2.

FIG. 49 is a diagram showing data on some of the X-ray detector channels in the multi-row X-ray detector 24 or two-dimensional X-ray area detector 24.

FIG. 50 is a diagram showing contour lines in the case of four-point interpolation.

FIG. 51 is a diagram showing contour lines in the case of three-point interpolation.

### DETAILED DESCRIPTION OF THE INVENTION

The present invention will be described in further detail with reference to modes for carrying it out illustrated in drawings. Incidentally, this is nothing to limit the invention.

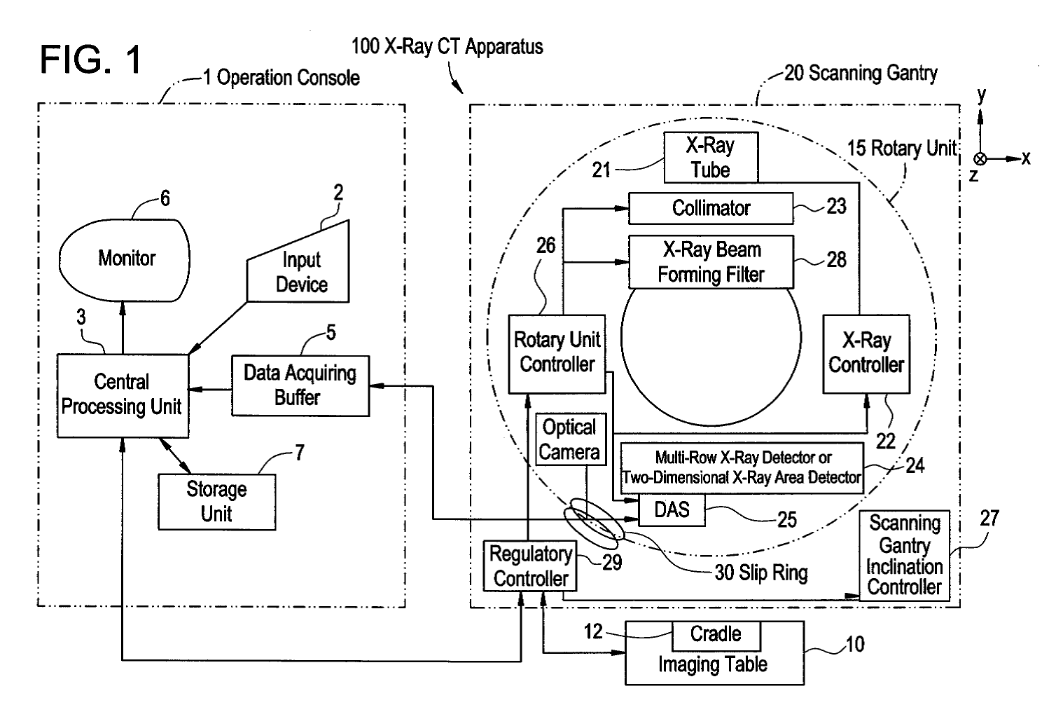

FIG. 1 is a configurative block diagram of an X-ray CT apparatus in one mode for carrying out the present invention. This X-ray CT apparatus 100 is equipped with an operation console 1, an imaging table 10 and a scanning gantry 20.

The operation console 1 is equipped with an input device 2 for accepting inputs by the operator, a central processing unit 3 for executing pre-treatments, image reconstruction processing, post-treatments and the like, a data acquisition buffer 5 for acquiring projection data collected by the scanning gantry 20, a monitor 6 for displaying tomograms reconstructed from projection data obtained by pre-treating X-ray detector data, and a storage unit 7 for storing programs, X-ray detector data, projection data and X-ray tomograms.

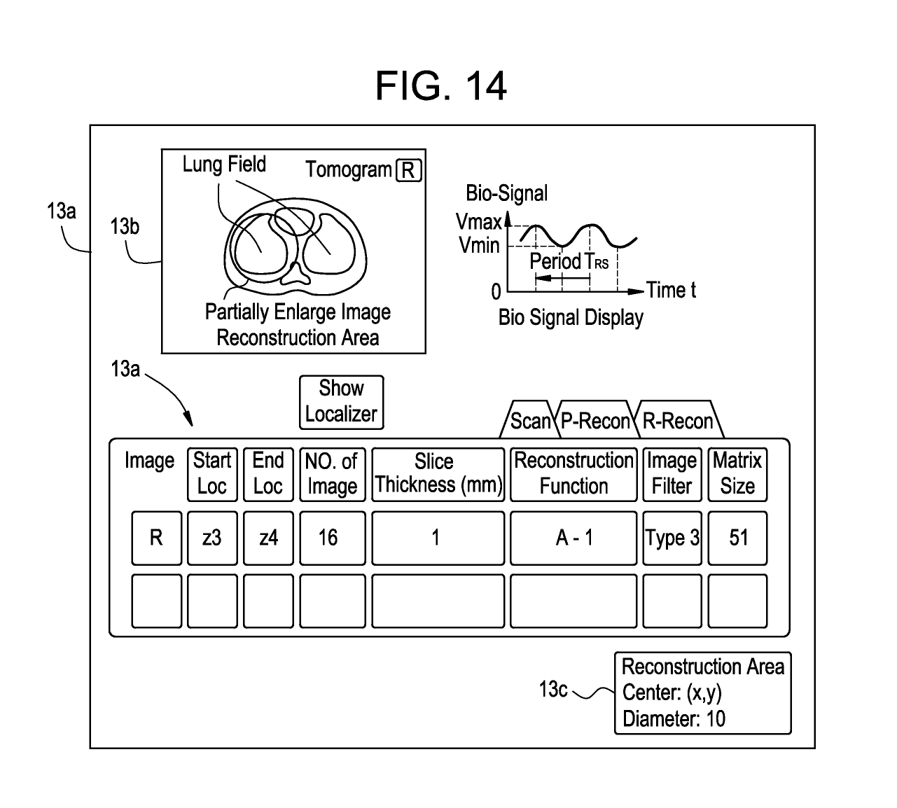

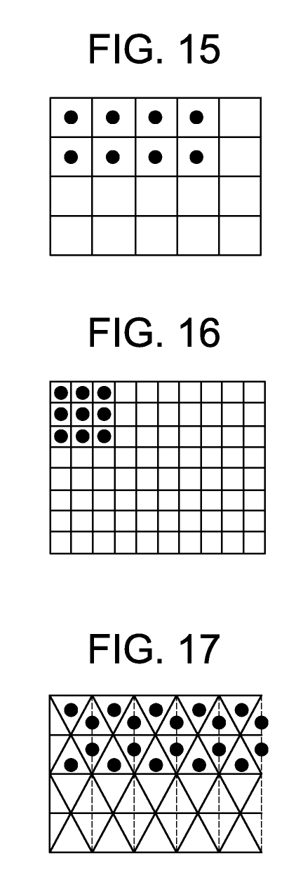

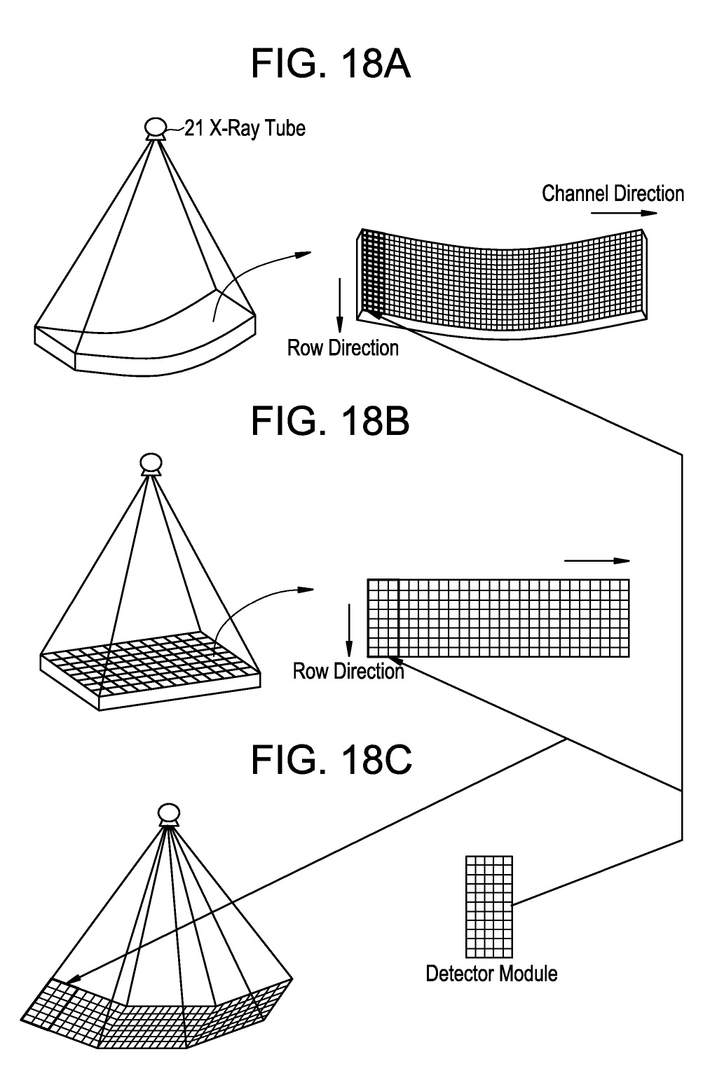

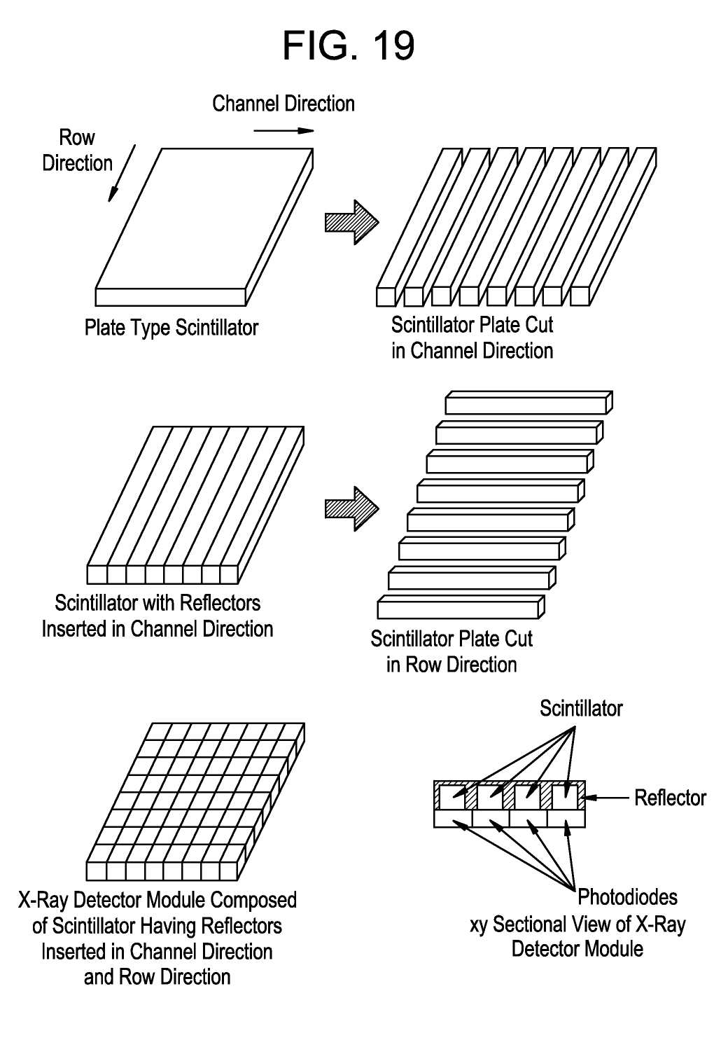

Imaging conditions are inputted through this input device 2 and stored in the storage unit 7. FIG. 14 shows an example of input screen of imaging conditions.

The imaging table 10 is equipped with a cradle 12. The cradle 12 places in and out a subject through the opening of the scanning gantry 20, with the subject being mounted on the cradle 12. The cradle 12 is lifted, lowered and moved along the table line by a motor built into the imaging table 10.

The scanning gantry 20 is equipped with an X-ray tube 21, an X-ray controller 22, a collimator 23, an X-ray beam forming filter 28, a multi-row X-ray detector 24, a DAS (Data Acquisition System) 25, a rotary unit controller 26 for controlling the X-ray tube 21 and others rotating around the body axis of the subject, and a regulatory controller 29 for exchanging control signals and the like with the operation console 1 and the imaging table 10. The X-ray beam forming filter 28 is an X-ray filter which is the least in filter thickness in the direction of X-rays toward the rotation center, which is the center of imaging, and increases in filter thickness toward the peripheries to enable more of X-rays to be absorbed. For this reason, exposure of the body surface of a subject whose sectional shape is close to a circle or an oval to radiation can be reduced. Further, the scanning gantry 20 can be inclined ahead of or behind the z-direction by approximately ±30 degrees by a scanning gantry inclination controller 27.

The X-ray tube 21 and the multi-row X-ray detector 24 turns around the rotation center IC. The vertical direction being supposed to be the y direction, the horizontal direction the x direction and the direction of the table and cradle movement perpendicular to them the z direction, the rotational plane of the X-ray tube 21 and the multi-row X-ray detector 24 is the xy plane. Further, the moving direction of the cradle 12 is the z direction.

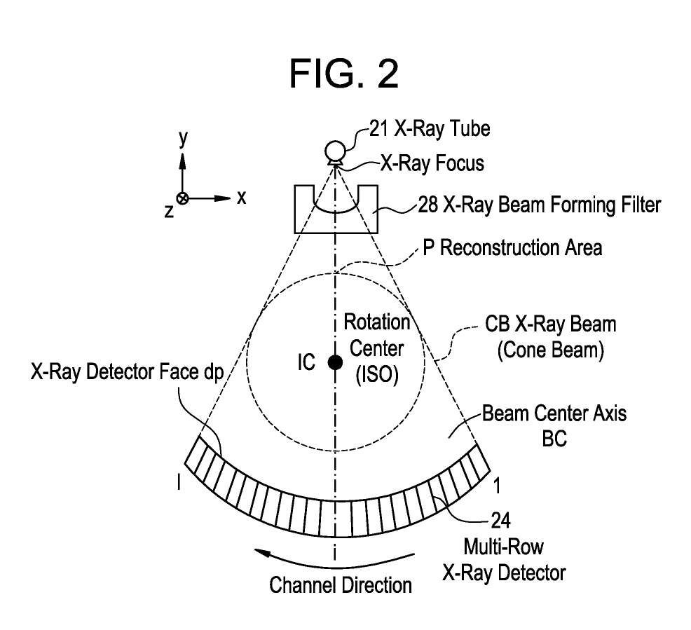

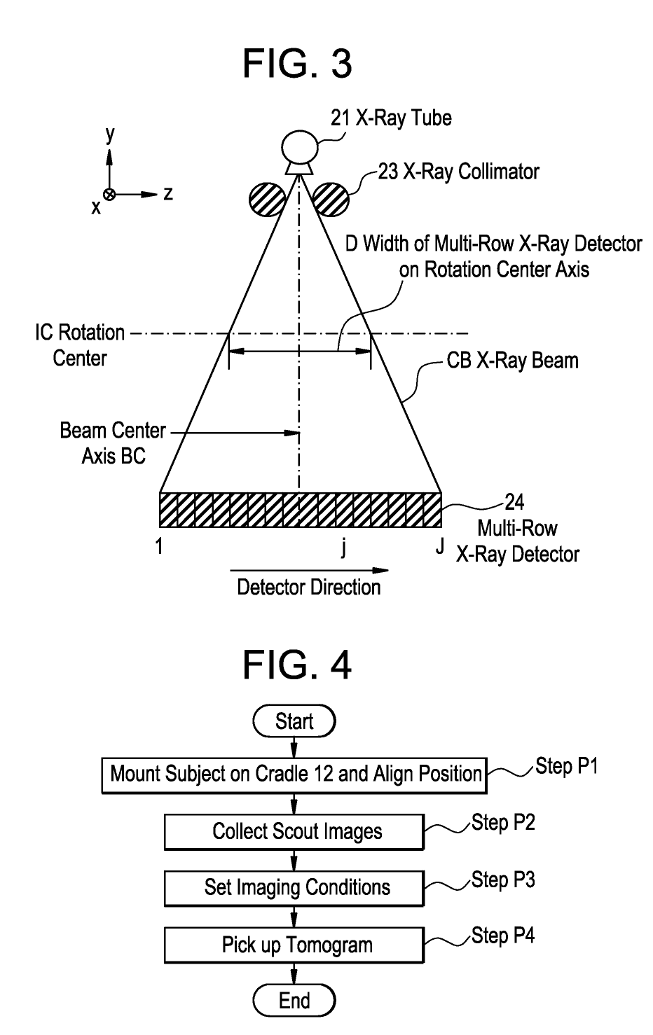

FIG. 2 and FIG. 3 show views of the geometrical arrangement of the X-ray tube 21 and the multi-row X-ray detector 24 as seen from the xy plane or the yz plane.

The X-ray tube 21 generates an X-ray beam known as cone beam CB. When the direction of the center axis of the cone beam CB is parallel to the y direction, the view angle is supposed to be 0 degree.

The multi-row X-ray detector 24 has, for instance, 256 detector rows in the z direction. Each X-ray detector row has, for instance, 1024 X-ray detector channels.

As shown in FIG. 2, after an X-ray beam leaving the X-ray focus of the X-ray tube 21 undergoes such spatial control by the X-ray beam forming filter 28 that more X-rays irradiate the center of the reconstruction area P and less X-rays irradiate the peripheries of the reconstruction area P, X-rays present within the reconstruction area P are absorbed by the subject, and transmitted X-rays are collected by the multi-row X-ray detector 24 as X-ray detector data.

As shown in FIG. 3, the X-ray beam leaving the X-ray focus of the X-ray tube 21 undergoes control by the X-ray collimator 23 in the slice thickness direction of the tomogram, namely in such a way that the X-ray beam width is D on the rotation center axis IC, and X-rays are absorbed by the subject present near the rotation center axis IC, and transmitted X-rays are collected by the multi-row X-ray detector 24 as X-ray detector data.

Collected projection data following irradiation with X-rays are supplied from the multi-row X-ray detector 24 and subjected to A/D conversion by the DAS 25, and inputted to the data acquisition buffer 5 via a slip ring 30. The data inputted to the data acquisition buffer 5 are processed by the central processing unit 3 in accordance with a program in the storage unit 7 to be reconstructed into a tomogram, which is displayed on the monitor 6.

The X-ray detector according to this embodiment realizes a high-resolution X-ray detector which can be fabricated in a simple process. By subjecting the high-resolution X-ray projection data to image reconstruction, a high-resolution tomogram can be obtained.

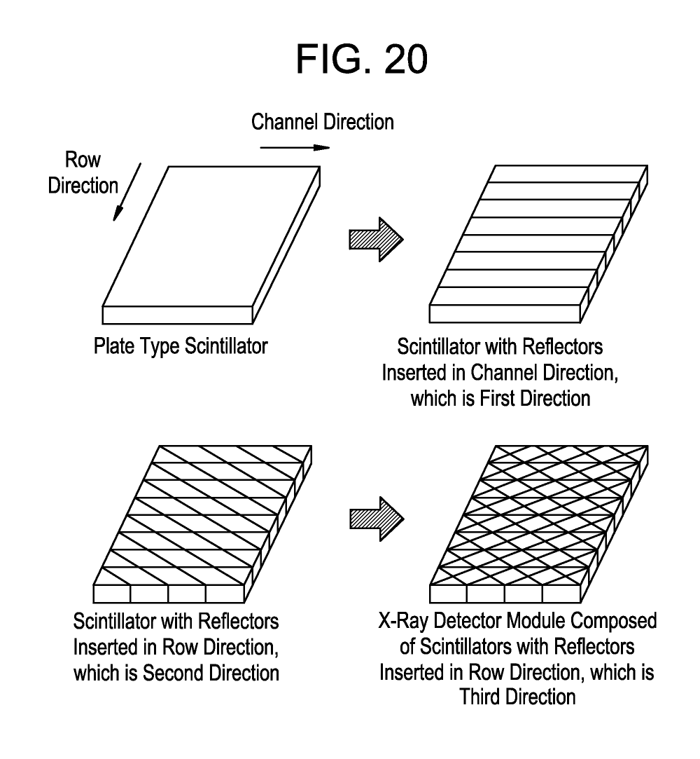

As shown in FIG. 20, a plate type scintillator is first cut in the row direction, which is the first direction, and the cut faces are painted with a reflector to suppress crosstalk by optical signals in each row direction. These rod-shaped pieces of scintillator painted with the reflector are combined again. After that, they are cut in the second direction, and rod-shaped pieces of the cut scintillator are painted with the reflector and combined again. After that, they are cut in the third direction, and rod-shaped pieces of the cut scintillator are painted with the reflector and combined again. The multi-row X-ray detector 24 or two-dimensional X-ray area detector 24 thereby fabricated has an X-ray detector structure in which each detector channel has a triangular shape as shown in FIG. 17.

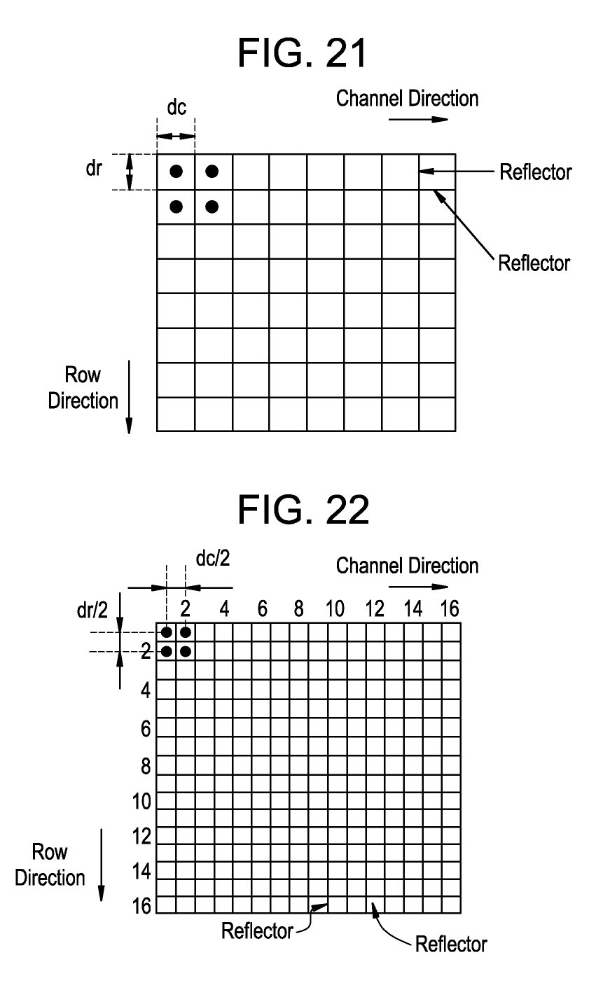

An example of conventional X-ray detector module is shown in FIG. 21. This X-ray detector module, which is an X-ray detector module having eight channels in the channel direction and eight channels in the row direction, can realize a multi-row X-ray detector 24. The intervals in the channel direction in this case are represented by dc, and those in the row direction, by dr. An X-ray detector module which derives from an attempt to raise the spatial resolution of this X-ray detector module shown in FIG. 21 both in the channel direction and in the row direction is shown in FIG. 22.

As shown in FIG. 22, the X-ray detector module has 16 channels in the channel direction and 16 channels in the row direction. The intervals between the X-ray detectors are dc/2 in the channel direction and are dr/2 in the row direction.

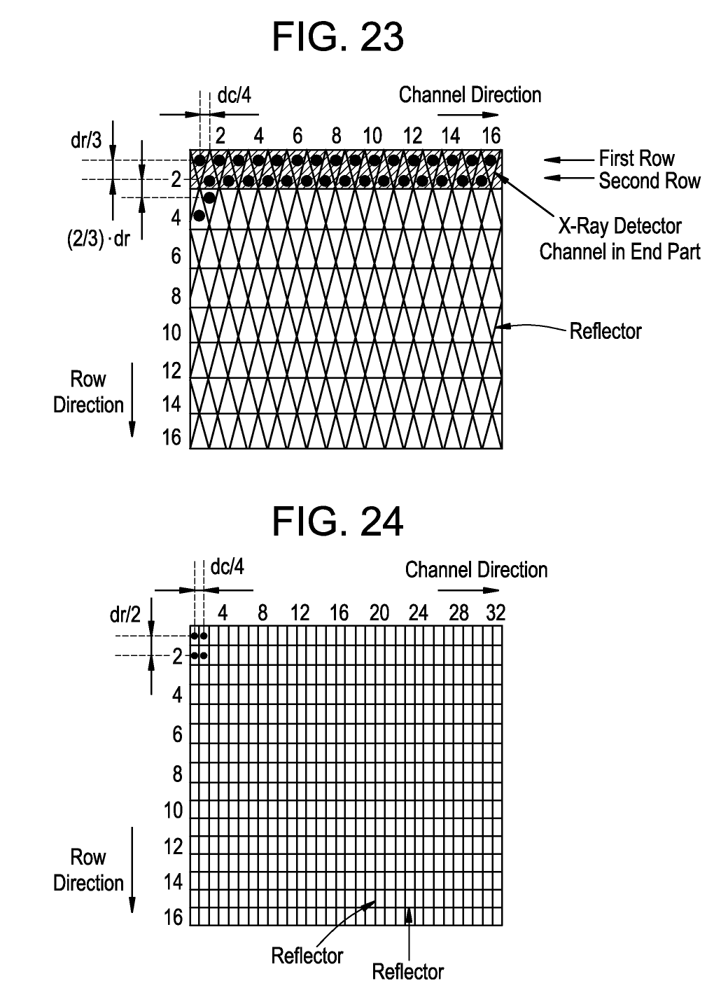

In this embodiment, by contrast, the intervals are dc/4 in the channel direction and dr/3 or (2/3)·dr in the row direction as shown in FIG. 23.

As shown in FIG. 24, the X-ray detector module has 32 channels in the channel direction and 16 channels in the row direction. In this case, the intervals between the X-ray detectors are dc/4 in the channel direction and dr/2 in the row direction.

Accordingly, the X-ray detector module of FIG. 23 is presumably positioned between the 16 (16 X-ray detector module of FIG. 22 and the 32 (32 X-ray detector module of FIG. 24 in spatial resolution.

Thus the X-ray detector module of FIG. 23, since its X-ray detector channels are appropriately dispersed in a two-dimensional space, a higher spatial resolution than the X-ray detector module of FIG. 22 can be expected.

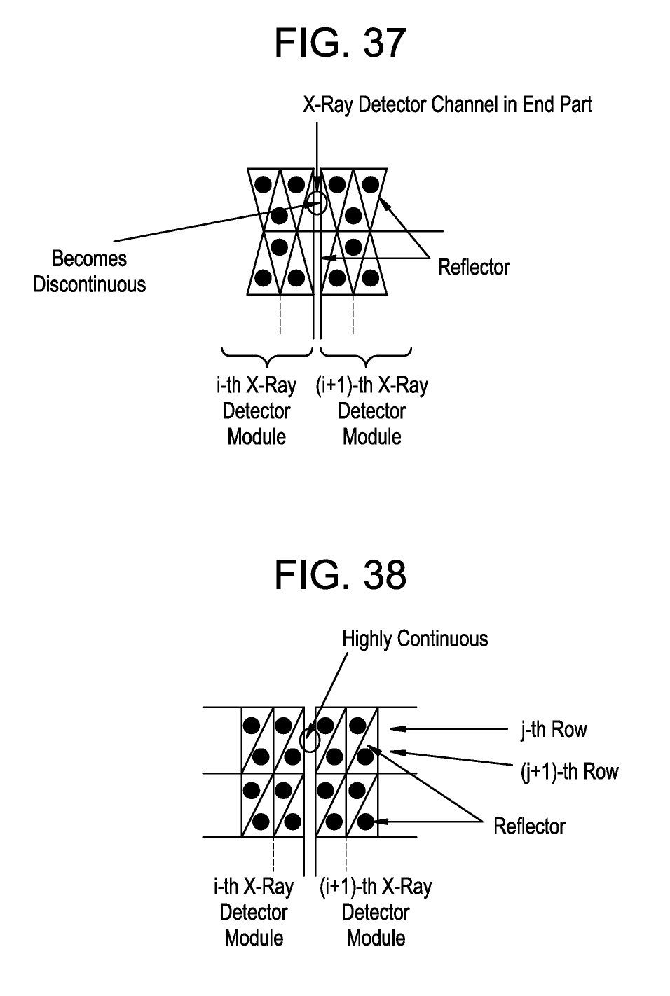

Further in the arrangement of FIG. 23, since the second direction and the third direction respectively are not parallel and perpendicular to the X-ray detector module in FIG. 20, the X-ray detector channel in the end part is ½ in area compared with other inner X-ray detector channels, and this poses a handling difficulty in respect of the continuity of all the X-ray detector channels. Usually, the end faces of an X-ray detector module both in the channel direction and in the row direction are painted with a reflector. As a result, the continuity of X-ray detectors is deteriorated because the reflector comes in between adjoining X-ray detector modules, between an X-ray detector channel in an end part and the adjoining X-ray detector module as represented by Example 1 of adjoining X-ray detector modules shown in FIG. 37. Improvements in this respect are achieved in the case illustrated in FIG. 36 and that illustrated in FIG. 38.

As represented by Example 2 of adjoining X-ray detector modules shown in FIG. 38, the X-ray detector channel in an end part is the same as another inside X-ray detector channel both in shape and area. The reflector on the end face of the X-ray detector module in the channel direction positioned between adjoining X-ray detector modules poses no problem to the continuity of X-ray detector channels. However, while the example of FIG. 23 manifests an exact hound's tooth check, the example shown in FIG. 36 and that shown in FIG. 38 do not show an exact hound's tooth check in the j-th row and the (j+1)-th row, forming shapes slightly inclined one of the channel directions.

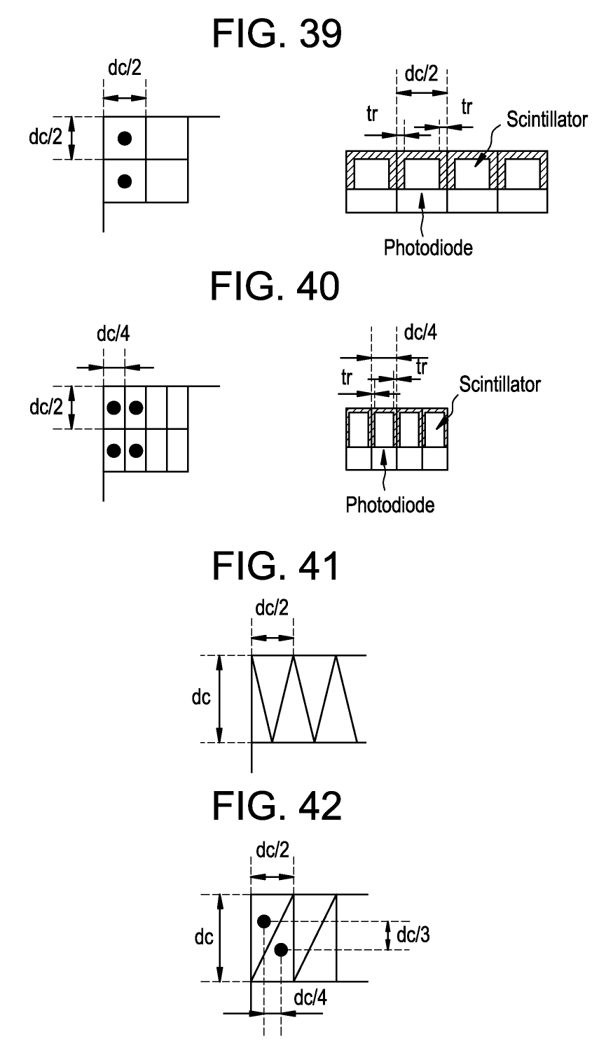

Further, the volume rates of the reflector in the channel direction and the row direction are considered regarding the 16-channel 16-row X-ray detector module of FIG. 39 and the 32-channel 16-row X-ray detector module of FIG. 40. Incidentally, all the quantities of the reflector on the X-ray detector surface (on the X-ray focus side) are assumed to be common quantities and accordingly are not considered here. In FIG. 39, the reflector is present in the following quantity in the X-ray detector area of (dc/2)².
4·dc/2·ιr=2·dc·ιr

The volume rates of the reflector in the channel direction and the row direction are as follows.
(2·dc·ιr)/(dc/2)²=8·ιr/dc

In FIG. 40, in the X-ray detector area of (dc/2)·(dc/4)=dc²/8,
(2·dc/2+2·dc/4)·ιr3/2·dc·ιr

The volume rates of the reflector in the channel direction and the row direction are as follows.
(3/2·dc·ιr)/dc²/8=12·ιr/dc

By contrast in FIG. 42, in the X-ray detector area of dc·dc/2=dc²/2,
(2·dc+2·dc/2+2.5^{1/2}dc/2)·ιr=(3+5^{1/2})dc·ιr

The volume rates of the reflector in the channel direction and the row direction are as follows.

$$\begin{matrix}( ( 3 + {5}^{1 / 2} ) dc \cdot ι r ) / ( {dc}^{2} \text{/} 2 ) = ( 6 + {2.5}^{1 / 2} ) ι r / dc \\ = 10.472 ι r / dc\end{matrix}$$

Similarly in FIG. 41, in the X-ray detector area of dc·dc/2=dc²/2,
(2·dc/2+4.17^{1/2}·dc/4)·ιr=(1+17^{1/2})dc·ιr

The volume rates of the reflector in the channel direction and the row direction are as follows.

$$\begin{matrix}( ( 1 + 2 \cdot {17}^{1 / 2} ) dc \cdot ι r / ( {dc}^{2} \text{/} 2 ) = ( 2 + 2 \cdot {17}^{1 / 2} ) dc \cdot ι r \\ = 10.246 ι r / dc\end{matrix}$$

Thus, Example 1 and Example 2 of the 16-channel 16-row X-ray detector module of this embodiment shown in FIG. 41 and FIG. 42 can achieve a resolution equivalent to the 32-channel 16-row X-ray detector module of FIG. 40 in a smaller reflector volume rate; namely, it can detect X-rays with a higher X-ray capturing efficiency.

FIG. 4 is a flow chart outlining the operation of the X-ray CT apparatus of this embodiment.

At step P1, the subject is mounted on the cradle 12 and aligned. The subject mounted on the cradle 12 undergoes alignment of the reference point of each region to the central position of the slice light of the scanning gantry 20.

At step P2, scout images are collected. Scout images are usually picked up at 0 degree and 90 degree, but in some cases, for instance for the head, only 90-degree scout images are picked up. Details of scout imaging will be described afterwards.

At step P3, imaging conditions are set. Usually, imaging is performed while displaying the position and size of the tomogram to be imaged on the scout image. In this case, information on the total X-ray dose per round of helical scanning, variable-pitch helical scanning, conventional scanning (axial scanning) or cine-scanning is displayed. Further in cine-scanning, if the number of revolutions or time length is inputted, X-ray dose information for the number of revolutions or the time length inputted in that interest area will be displayed.

At step P4, tomography is performed. Details of the tomography will be described afterwards.

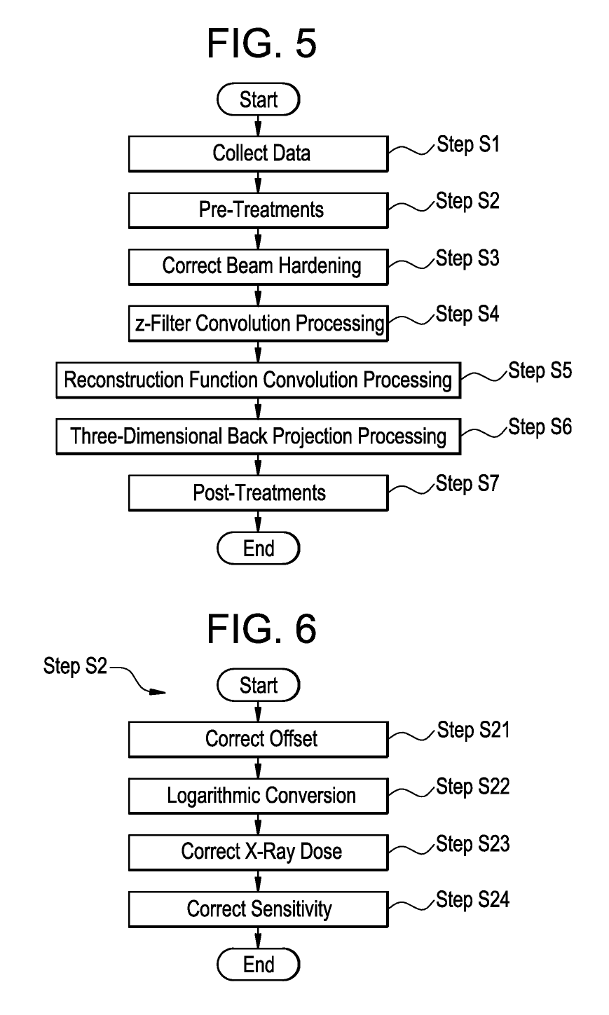

FIG. 5 is a flow chart outlining the operations of tomography and scout imaging by the X-ray CT apparatus 100 according to the invention.

At step S1, in helical scanning, X-ray detector data are collected while rotating the X-ray tube 21 and the multi-row X-ray detector 24 around the object of imaging and linearly moving the cradle 12 on the table 10, the X-ray detector being collected by adding the z-direction position z table (view) to X-ray detector data DO (view, j, i) represented by the view angle view, the detector row number j and the channel number i. In variable-pitch helical scanning, not only data collection in helical scanning is performed in a constant speed range but also data collection is carried out during acceleration and during deceleration.

Further, in conventional scanning (axial scanning) or cine-scanning, X-ray detector data are collected by rotating the data collection line one round or a plurality of rounds while keeping the cradle 12 on the imaging table 10 fixed in a certain z-direction position. X-ray detector data are further collected by rotating the data collection line one round or a plurality of rounds as required after moving to the next z-direction position.

On the other hand, in scout imaging, X-ray detector data are collected while keeping the X-ray tube 21 and the multi-row X-ray detector 24 fixed and linearly moving the cradle 12 on the imaging table 10.

At step S2, X-ray detector data D0 (view, j, i) are pre-treated to be converted into projection data. The pre-treatments comprise offset correction at step S21, logarithmic conversion at step S22, X-ray dose correction at step S23 and sensitivity correction at step S24 as shown in FIG. 6.

In scout imaging, by displaying the pre-treated X-ray detector data matched with the pixel size in the channel direction and the pixel size in the z-direction, which is the linear moving direction of the cradle, matched with the display pixel size of the monitor 6, the scout image is completed.

At step S3, the pre-treated projection data D1 (view, j, i) are subjected to beam hardening correction. The beam hardening correction at S3 can be expressed in, for instance, a polynomial form as represented below, with the projection data having undergone sensitivity correction at S24 of the pre-treatment S2 being represented by D1 (view, j, i) and the data after the beam hardening correction at S3 by D11 (view, j, i).

[Mathematical Expression 1]
D11(view, j,i)=D1(view, j,i)·(Bo(j,i)+B₁(j,i)·D1(view, j,i)+B₂(j,i)·D1(view, j,i)²)

Since each j rows of detectors can be subjected to beam hardening correction independently of others then, if the tube voltage of each data collection line differs from others depending on imaging conditions, differences in detector characteristics from row to row can be compensated for.

At step S4, the projection data D11 (view, j, i) having undergone beam hardening correction are subjected to filter convolution, by which filtering is done in the z direction (the row direction).

Thus, the data D11 (view, j, i) (i=1 to CH, j=1 to ROW) of the multi-row X-ray detector having undergone beam hardening correction after the pre-treatment at each view angle and on each data collection line are subjected to, for instance, filtering whose row-direction filter size is five rows.

[Mathematical Expression 2]
(w1(i), w2(i), w3(i), w4(i), w5(i)),
provided that

$${\sum }_{k - 1}^{5} {w}_{k} ( i ) = 1$$

The corrected detector data D12(view, j, i) will be as follows.

[Mathematical Expression 3]

$$D 12 ( view , j , i ) = {\sum }_{k - 1}^{5} ( D 11 ( view , j + k - 3 , i ) \cdot {w}_{k} ( j ) )$$

Incidentally, the maximum channel width being supposed to be CH and the maximum row value being ROW, the following will hold.

[Mathematical Expression 4]
D11(view, 1,i)=D11(view, 0,i)=D11(view, 1,i)
D11(view, ROW, i)=D11(view, ROW+1, i)=D11(view, ROW+2, i)

On the other hand, the slice thickness can be controlled according to the distance from the center of image reconstruction by varying the row-direction filter coefficient from channel to channel. Since the slice thickness is usually greater in the peripheries than at the center of reconstruction in a tomogram, the slice thickness can be made substantially uniform whether in the peripheries or at the center of image reconstruction by so differentiating the row-direction filter coefficient between the central part and the peripheries that the range of the row-direction filter coefficient is varied more greatly in the vicinities of the central channel and varied more narrowly in the vicinities of the peripheral channel.

By controlling the row-direction filter coefficient between the central channels and the peripheral channels of the multi-row X-ray detector 24 in this way, the control of the slice thickness can also be differentiated between the central part and the peripheries. By slightly increasing the slice thickness with the row-direction filter, both artifact and noise can be substantially improved. The extent of improvement of artifact and that of noise can be thereby controlled. In other words, a tomogram having undergone three-dimensional image reconstruction, namely picture quality in the xy plane, can be controlled. Another possible embodiment, a tomogram of a thin slice thickness can be realized by using deconvolution filtering for the row-direction (z-direction) filter coefficient.

Further, X-ray projection data of the fan beam are converted into X-ray projection data of the parallel beam.

At step S5, convolution of the reconstructive function is performed. Thus, the result of Fourier transform is multiplied by the reconstructive function to achieve inverse Fourier transform. In the convolution of reconstructive function at S5, data after the convolution of z-filter being represented by D12, data after the convolution of reconstructive function by D13 and the reconstructive function to be convoluted by Kernel (j), the processing to convolute the reconstructive function can be expressed in the following way.

[Mathematical Expression 5]
D13(view, j,i)=D12(view, j,i)*Kernel(j)

Thus, since the reconstructive function Kernel (j) permits independent convolution of the reconstructive function on each j rows of detectors, differences in noise characteristics and resolution characteristics from one row to another can be compensated for.

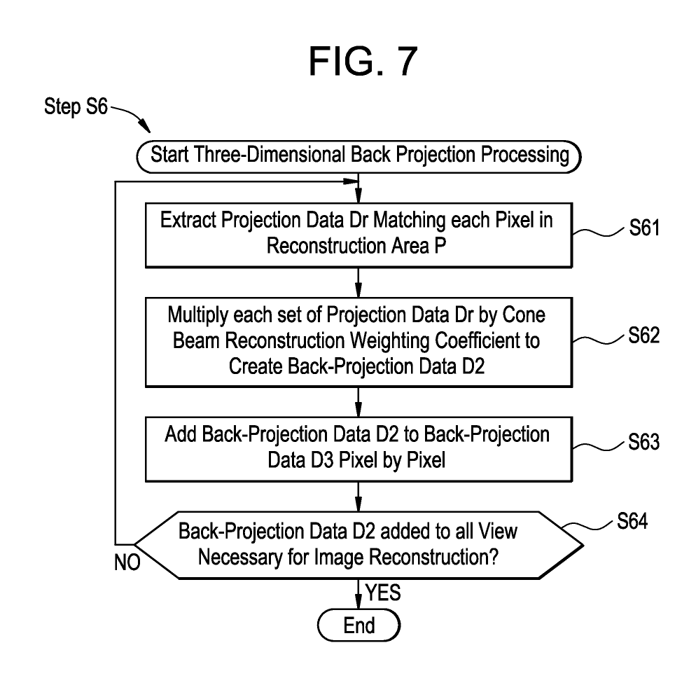

At step S6, the projection data D13 (view, j, i) having undergone convolution of the reconstructive function are subjected to three-dimensional back-projection to obtain back-projected data D3 (x, y, z). The image to be reconstructed is reconstructed into a three-dimensional image on a plane perpendicular to the z-axis, the xy plane. The following reconstruction area P is supposed to be parallel to the xy plane. This three-dimensional back-projection will be described afterwards with reference to FIG. 7.

At step S7, the back-projected data D3 (x, y, z) are subjected to post-treatments including image filter convolution and CT value conversion to obtain a tomogram D31 (x, y).

In the image filter convolution as post-treatment, with the data having gone through three-dimensional back-projection being represented by D31 (x, y, z), the data having gone through image filter convolution by D32 (x, y, z) and the image filter by Filter (z):

[Mathematical Expression 6]
D32(x, y, z)=D31(x, y, z)*Filter(z)

Thus, since independent image filter convolution is possible on each j rows of detectors, differences in noise characteristics and resolution characteristics from one row to another can be compensated for.

The tomogram that is obtained is displayed on the monitor 6.

FIG. 7 is a flow chart showing details of the three-dimensional back-projection processing (step S6 in FIG. 5).

In this embodiment, the image to be reconstructed is reconstructed into a three-dimensional image on a plane perpendicular to the z-axis and the xy plane. The following reconstruction area P is supposed to be parallel to the xy plane.

At step S61, note is taken on one view out of all the views needed for image reconstruction of a tomogram (namely 360-degree views or “180-degree +fan angle” views), and projection data Dr corresponding to the pixels in the reconstruction area P are extracted.

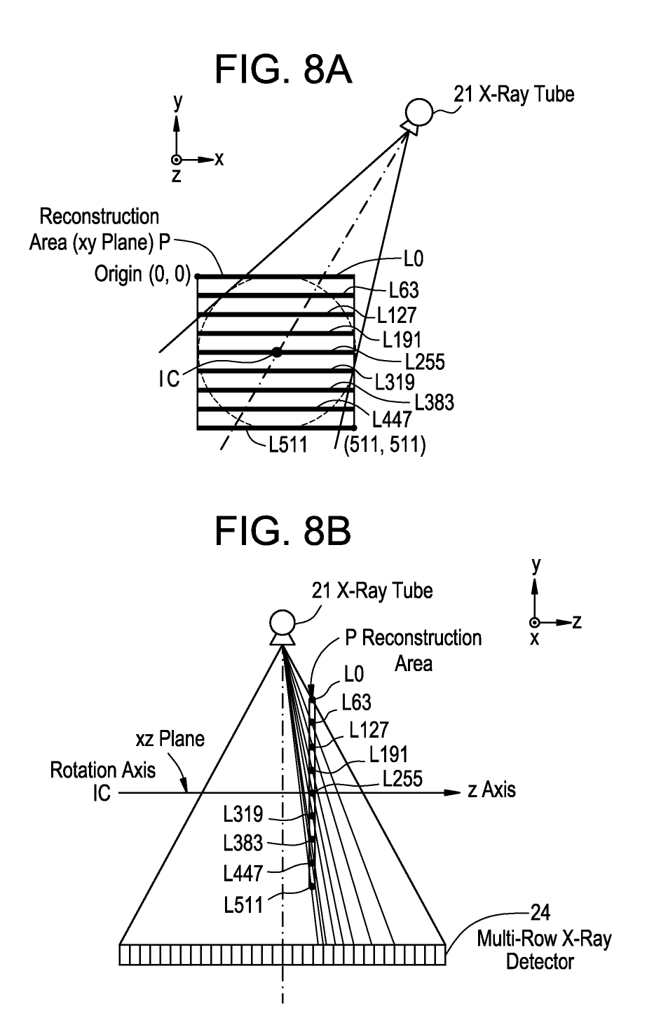

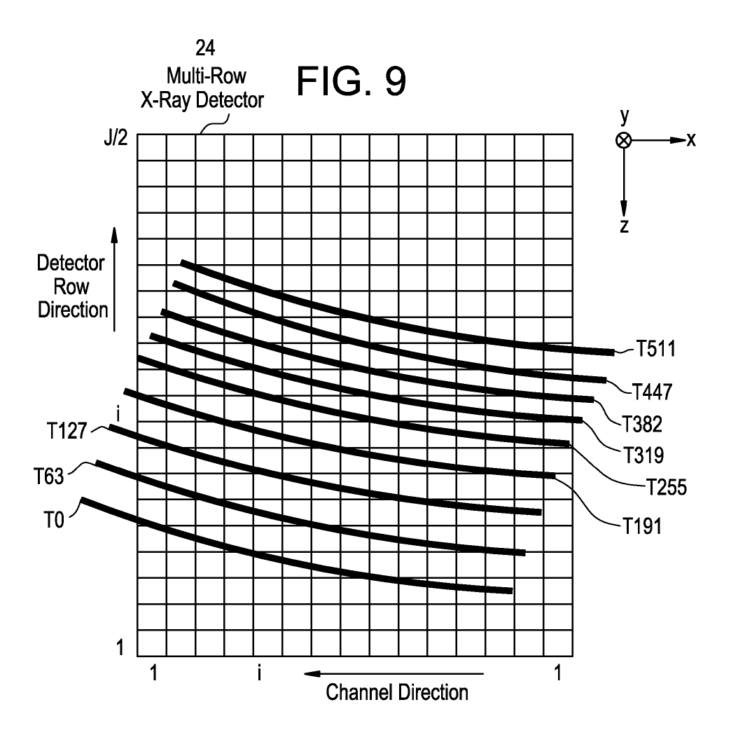

As shown in FIG. 8(a) and FIG. 8(b), a square area of 512×512 pixels parallel to the xy plane being supposed to be the reconstruction area P, and a pixel row L0 of y=0, a pixel row L63 of y=63, a pixel row L127 of y=127, a pixel row L191 of y=191, a pixel row L255 of y=255, a pixel row L319 of y=319, a pixel row L383 of y=383, a pixel row L447 of y=447 and a pixel row L511 of y=511, all parallel to the x-axis of y=0, being taken as rows, if projection data on lines T0 through T511 are extracted as shown in FIG. 9, wherein these pixel rows L0 through L511 are projected on the plane of the multi-row X-ray detector 24 in the X-ray transmitting direction, they will constitute projection data Dr (view, x, y) of pixel rows L0 through L511. It is provided, however, that x and y match pixels (x, y) in the tomogram.

To add, since X-ray detectors in the multi-row X-ray detector 24 or two-dimensional X-ray area detector 24 of this embodiment are not X-ray detectors having a usual square lattice or rectangular lattice structure, some contrivance is needed not to let the resolution drop in the extraction of X-ray projection data in the three-dimensional back-projection processing of this embodiment. This contrivance not to let the resolution drop will be described afterwards.

Whereas the X-ray transmitting direction is determined by the geometrical positions of the X-ray focus of the X-ray tube 21, the pixels and the multi-row X-ray detector 24, since the z-coordinate z (view) of the X-ray detector data D0 (view, j, i) is known as the z-direction of the linear table movement Z table (view) attached to the X-ray detector data, the X-ray transmitting direction can be accurately figured out in the data collection geometric system of the X-ray focus and the multi-row X-ray detector even if the X-ray detector data D0 (view, j, i) are obtained during acceleration or deceleration.

Incidentally, if part of the lines goes out of the channel direction of the multi-row X-ray detector 24 as does, for instance, the line T0 resulting from the projection of the pixel row L0 onto the plane in the multi-row X-ray detector 24 in the X-ray transmitting direction, the matching projection data Dr(view, x, y) are set to “0”. If they go out of the z-direction, it will be figured out by extrapolating projection data Dr (view, x, y).

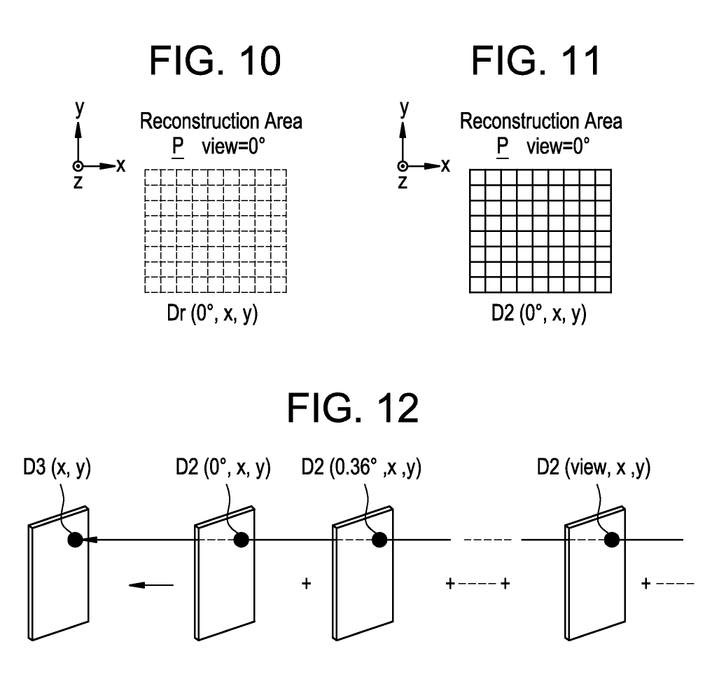

In this way, projection data Dr (view, x, y) matching the pixels of the reconstruction area P can be extracted as shown in FIG. 10.

Referring back to FIG. 7, at step S62, projection data Dr (view, x. y) are multiplied by a cone beam reconstruction weighting coefficient to create projection data D2 (view, x, y) shown in FIG. 11.

The cone beam reconstruction weighting coefficient w (i, j) here is as follows. In reconstructing a fan beam image, the following relationship holds where γ is the angle which a straight line linking the focus of the X-ray tube 21 and a pixel g (x, y) forms with respect to the center axis Bc of the X-ray beam where view=βa and the view opposite thereto is view=β:
βb=βa+180°+2γ

With the angles formed by the X-ray beam passing the pixel g (x, y) on the reconstruction area P and the X-ray beam opposite thereto with respect to the reconstruction plane P being respectively represented by αa and αb, the back-projected pixel data D2 (0, x, y) are figured out by adding after multiplication with reconstruction weighting coefficients ωa and ωb. In this case, the following holds.

[Mathematical Expression 7]
D2(0, x, y)=ωa·D2(0, x, y)_{—}a+ωb·D2(0, x, y)_{—}b
where D2 (0, x, y)_a are supposed to be the projected data of view βa and D2 (0, x, y)_b, the projected data of view βb.

Incidentally, the sum of the mutually opposite beams of cone beam reconstruction weighting coefficients is:
ωa+ωb=1

By adding the products of multiplication by cone beam reconstruction weighting coefficients ωa and ωb, the cone angle artifact can be reduced.

For instance, reconstruction weighting coefficients ωa and ωb obtained by the following formulas can be used. In these formulas, ga is the weighting coefficient of the view βa and gb, the weighting coefficient of the view βb.

Where ½ of the fan beam angle is γmax, the following holds.

[Mathematical Expression 8]
ga=f(γmax, αa, βa)
gb=f(γmax, αa, βb)
xa=2·ga^{q}/(ga^{q}+gb^{q})
xb=2gb^{q}/(ga^{q}+gb^{q})
wa=xa²·(3−2xa)
wb=xb²·(3−2xb)

(For instance, q=1 is supposed.)

For instance, if max[ ] is supposed to be a function taking up what is greater in value as an example of ga and gb, the following will hold.

[Mathematical Expression 9]
ga=max└0, {(π/2+γmax)−|βa|}┘·|tan(αa)|
gb=max[0, {(π/2+γmax)−, |βb|}]·|tan(αb)|

In the case of fan beam image reconstruction, each pixel of the reconstruction area P is further multiplied by a distance coefficient. The distance coefficient is (r1/r0)² where r0 is the distance from the focus of the X-ray tube 21 to the detector row j and the channel i of the multi-row X-ray detector 24 matching the projection data Dr, and r1 is the distance from the focus of the X-ray tube 21 to a pixel matching the projection data Dr on the reconstruction area P.

In the case of parallel beam image reconstruction, it is sufficient to multiply each pixel of the reconstruction area P only by the cone beam reconstruction weighting coefficient w (i, j).

At step S63, projection data D2 (view, x, y) are added, correspondingly to pixels, to back-projected data D3 (x, y) cleared in advance as shown in FIG. 12.

At step S64, steps 61 through S63 are repeated for all the views necessary for CT image reconstruction (namely 360-degree views or “180-degree+fan angle” views) to obtain back-projected data D3(x, y) as shown in FIG. 12.

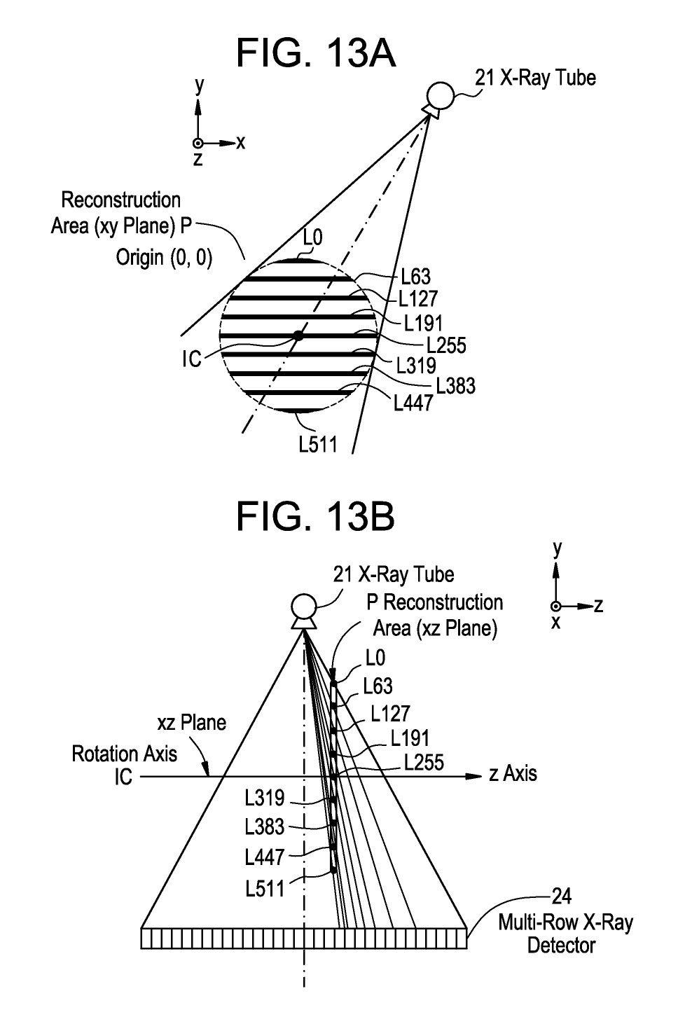

Incidentally, the reconstruction area P may as well be a circular area of 512 pixels in diameter as shown in FIG. 13(a) and FIG. 13(b) in stead of a square area of 512×512 pixels.

The foregoing described the overall flow including X-ray data collection, pre-treatment and back projection processing in this embodiment. In the following, back projection processing to prevent resolution from deteriorating in the image reconstruction processing in this embodiment will be described in further detail.

First with respect to Embodiment 1, a case in which data are collected by the multi-row X-ray detector 24 or two-dimensional X-ray area detector 24 using Example 1 of X-ray detector module of the embodiment shown in FIG. 23 will be described.

Then with respect to Embodiment 2, a case in which Example 2 of X-ray detector module of the embodiment shown in FIG. 36 is used will be described.

Further with respect to Embodiment 3, a case in which resolution in the channel direction is enhanced to improve the spatial resolution of tomograms by interleaving X-ray detector data of adjoining rows will be described.

### Embodiment 1

With respect to Embodiment 1, a case in which data are collected by the multi-row X-ray detector 24 or two-dimensional X-ray area detector 24 using the X-ray detector module shown in FIG. 23 will be described.

In this embodiment, since data are collected by the multi-row X-ray detector 24 or two-dimensional X-ray area detector 24 using the X-ray detector module shown in FIG. 23, X-ray detector data which look as if resulting from X-ray data collection by X-ray detectors in a hound's tooth check pattern can be collected.

The pre-treatments and reconstruction function convolution processing in this case may consists of the pre-treatments at step S2 of FIG. 5 as described above, and the beam hardening correction at step S3, Z-filter convolution processing at step S4, reconstruction function convolution processing at step S5 and the post-treatments at step S7 may be carried out similarly.

Further in the image reconstruction of three-dimensional back projection processing at step S6, three-dimensional back projection processing is performed from projection data of a hound's tooth check structure in which the even number rows and the odd number rows are off each other by half of the channel-direction spacing dc of X-ray detectors in the channel direction, namely by dc/2, and off by dr/3 or (2/3)·dr in the row direction as shown in FIG. 23.

If in this case four points of the hound's tooth check pattern are taken up as shown in FIG. 28, the distance to the actual projection data will be elongated, and the weighted addition will blur thee projection data.

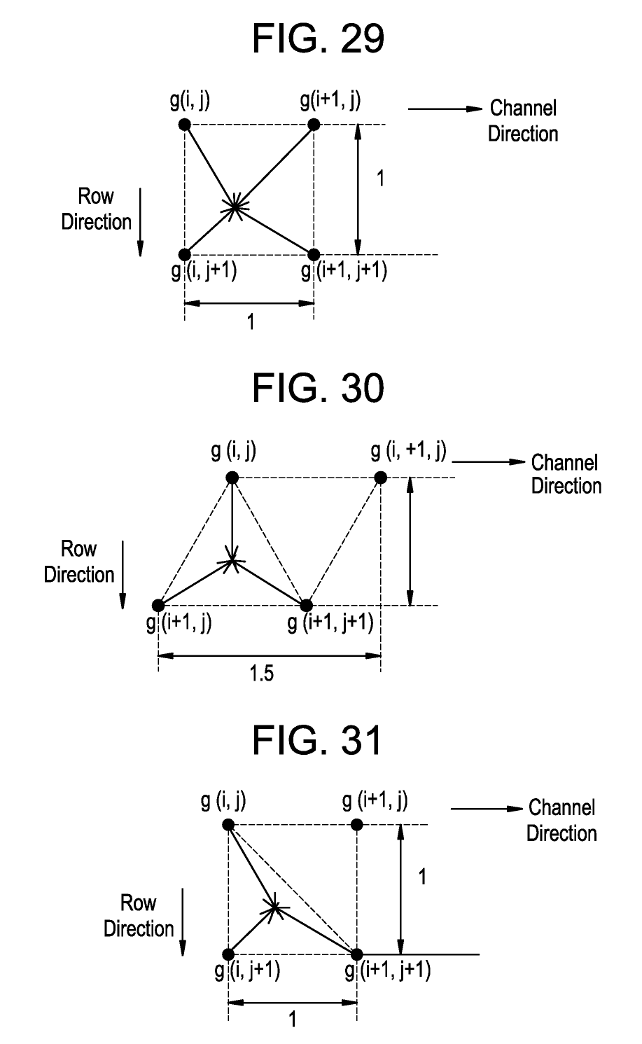

Usually when the multi-row X-ray detector 24 or two-dimensional X-ray area detector 24 collects X-ray projection data from all the rows of X-ray detectors in a square lattice structure at the same timing, data obtained weighted addition of positions indicated by “x” as shown in FIG. 29 are figured by weighted addition from four nearby points, namely four points of the actual data of projection data in the positions indicated by “●”. The length in the channel direction and the row direction of one mesh of the square lattice structure of the multi-row X-ray detector 24 or two-dimensional X-ray area detector being represented by “1”, the distance blurred by the weighted addition in this case is “1” both in the channel direction and in the row direction.

Figuring out data by weighted addition processing from X-ray projection data in the hound's tooth check arrangement on the extension of this idea will prove to figuring out the data by subjecting the four apexes of a parallelogram extending in the channel direction as shown in FIG. 28 to weighted addition processing. In this case, the X-ray projection data will be blurred in the channel direction, and the tomogram that is finally obtained will also be blurred, resulting in deteriorated spatial resolution. The distanced blurred by weighted addition in this case will be “1.5” in the channel direction and “1” in the row direction.

In view of this, three-point weighted addition processing of three selected points near apexes of a parallelogram as shown in FIG. 30 makes possible weighted addition processing less susceptible to blurring of projection data than four-point weighted addition processing. In this case the distance blurred by weighted addition is “0.5” in the channel direction and “1” in the row direction.

A similar effect can be achieved by using X-ray projection data of a square lattice structure in this three-point weighted addition as shown in FIG. 31. In this case, too, the distance blurred by weighted addition is “0.5” in the channel direction and “1” in the row direction.

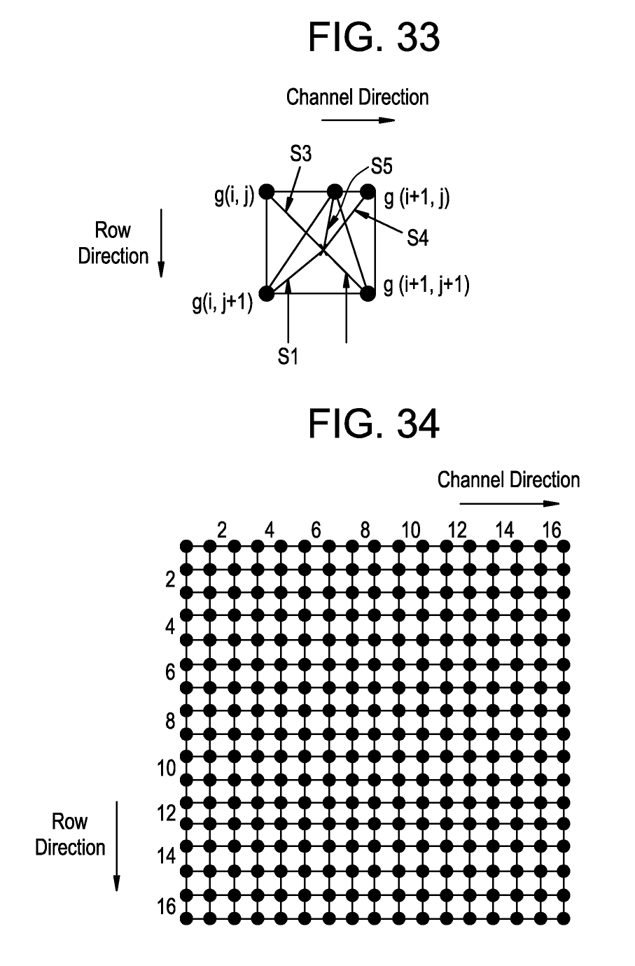

For another explanation of the reduced blurring of projection data in three-point weighted addition processing, reference may be made to FIG. 33.

The distance to actual data in three-point weighted addition is L3=S1+S2+S5

The distance to actual data in four-point weighted addition is L4=S1+S2+S3+S4

Since S5 is smaller than whichever of S3 and S4, the following can be the obviously.
L4>L3

Therefore, three-point weighted addition can be considered less susceptible to blurring of projection data.

Returning to the description of three-point weighted addition of X-ray detectors in the hound's tooth check structure shown in FIG. 30, the real data of the X-ray projection data at the four points near the position of data to be figured out by weighted addition, gΔi+Δi, j+Δj) (where 0Δ≦Δi ≦1, 0≦(j≦1), as shown in FIG. 30 are supposed to be:
g(i, j), g(i+1, j), g(i, j+1), g(i+1, j+1)

To select three nearer points out of these four points:
(1) Where 0≦″i≦½, 0≦Δj≦½g(i, j), g(i+1, j), g(i, j+1) are selected.
(2) Where 0≦Δi<½, ½≦Δj≦1 g(i, j), g(i, j+1), g(i+1, j+1) are selected.
(3) Where ½<Δi<1,0<Δj<½g(i, j), g(i+1, j)·g(i+1, j+1) are selected.
(4) Where ½<Δi<1, ½<Δj<1 g(i+1, j), g(i, j+1), g(i+1, j+1) are selected.

Weighted addition is processed in the following way by multiplying the three points selected in this way by weighting coefficients.

[Mathematical Expression 10]
g(i+Δi, j+Δj)=wₐ·g(i, j)+w_{b}g(i+1, j)+w_{c}·g(i, j+1)wₐ+w_{b}+W_{c}=1

Whereas there are many ways to determine weighting coefficients wₐ, w_{b} and w_{c}, linear weighting coefficients (first-order weighting coefficients) are stated below as one example.

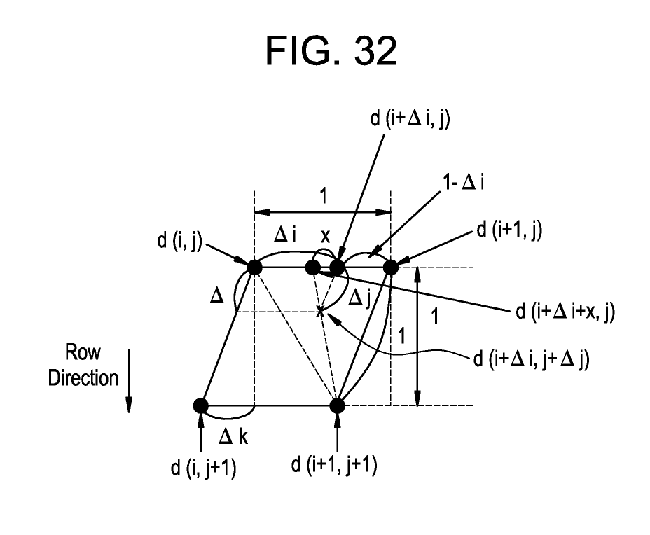

FIG. 32 shows a method of data extraction using three-point weighted addition processing by linear weighted addition.

[Mathematical Expression 11]
Δd(i+Δi+x, j)d(i+1, j)d(i+1, j+1)
Δd(i+Δi+x, j)d(i+Δi, j)d(i+Δi, j+Δj)

The similarity of the above gives the following relationship.

[Mathematical Expression 12]

$$\frac{x}{1 - Δ i + x} = \frac{Δ i}{1} \qquad ( Formula 1 )$$

From this, x can be figured out as follows.

[Mathematical Expression 13]

$$x = Δ j ( 1 - Δ i + x ) = Δ j ( 1 - Δ i ) + Δ j \cdot x \\ x \cdot ( 1 - Δ j ) = Δ j ( 1 - Δ i ) \\ x = \frac{1 - Δ i}{1 - Δ j} \cdot Δ j \qquad ( Formula 2 )$$

Incidentally, d(i+Δi+x, j) can be obtained by subjecting d(i, j) and d(i+1, j) to weighted addition processing in the following manner.

[Mathematical Expression 14]
d(i+Δi+x, j)=(1Δi+x)·d(i, j)+(Δi−x)d(i+1, j) (Formula5)

In this Formula 5, (1−Δi+x) and (i−x) can be obtained from (Formula 2) in the following manner.

[Mathematical Expression 15]

$$\begin{matrix}( 1 - Δ i + x ) = 1 - Δ j + \frac{1 - Δ i}{1 - Δ j} \cdot Δ j \\ = ( 1 - Δ i ) ( \frac{1 - Δ j + Δ j}{1 - Δ j} ) \\ = \frac{1 - Δ i}{1 - Δ j}\end{matrix} \qquad ( Formula 3 )$$

[Mathematical Expression 16]

$$\begin{matrix}( Δ i - x ) = Δ i - \frac{1 - Δ i}{1 - Δ j} \cdot Δ j \\ = \frac{Δ i - Δ i \cdot Δ j - Δ j + Δ i \cdot Δ j}{1 - Δ j} \\ = \frac{Δ i - Δ j}{1 - Δ j}\end{matrix} \qquad ( Formula 4 )$$

d(i+Δi, j+Δj) can be obtained from (Formula 5), (Formula 3) and (Formula 4) in the following manner.

[Mathematical Expression 17]

$$\begin{matrix}d ( i + Δ i , j + Δ j ) = \frac{Δ j}{\sqrt{1 + {( Δ k )}^{2}}} \cdot d ( i + 1 , j + 1 ) + \\ ( 1 - \frac{Δ j}{\sqrt{1 + {( Δ k )}^{2}}} ) \cdot d ( i + Δ i + x , j ) \\ = \frac{Δ j}{\sqrt{1 + {( Δ k )}^{2}}} \cdot d ( i + 1 , j + 1 ) + \\ ( 1 - \frac{Δ j}{\sqrt{1 + {( Δ k )}^{2}}} ) \cdot \\ ( \begin{matrix}( 1 - Δ i + x ) \cdot d ( i , j ) + \\ ( Δ i - x ) \cdot d ( i + 1 , j )\end{matrix} ) \\ = \frac{Δ j}{\sqrt{1 + {( Δ k )}^{2}}} \cdot d ( i + 1 , j + 1 ) + \\ ( 1 - \frac{Δ j}{\sqrt{1 + {( Δ k )}^{2}}} ) \cdot \\ ( \begin{matrix}\frac{1 - Δ i}{1 - Δ j} \cdot d ( i , j ) + \\ \frac{Δ i - Δ j}{1 - Δ j} \cdot d ( i + 1 , j )\end{matrix} ) \\ = \frac{Δ j}{\sqrt{1 + {( Δ k )}^{2}}} \cdot d ( i + 1 , j + 1 ) + \\ ( 1 - \frac{Δ j}{\sqrt{1 + {( Δ k )}^{2}}} ) \cdot \\ \frac{\begin{matrix}( 1 - Δ i ) \cdot d ( i , j ) + \\ ( Δ i - Δ j ) \cdot d ( i + 1 , j )\end{matrix}}{1 - Δ j}\end{matrix} \qquad ( Formula 6 )$$

In this way, data extraction using three-point weighted addition processing by linear weighted addition can be accomplished.

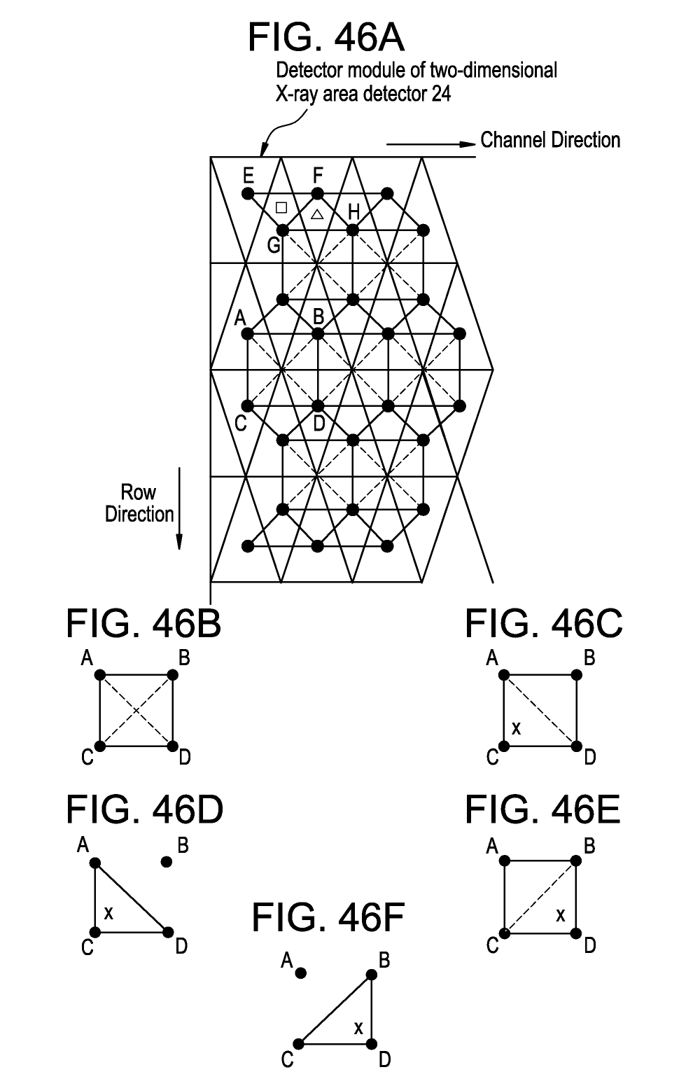

By using this data extraction method for the above-described three-dimensional back projection processing at step S6 of FIG. 5, data can be extracted by processing weighted addition without blurring data in the channel direction when data are extracted from X-ray projection data in a hound's tooth check arrangement in which the X-ray data collection is timed off each other between the even number rows and the odd number rows of the multi-row X-ray detector 24 or two-dimensional X-ray area detector 24, and tomograms of high resolution can be obtained without blurring pixel data even in tomograms from three-dimensional back projection processing.

Whereas the way of three points for three-point weighted addition processing or three-point interpolation processing in Embodiment 1 basically is “to select the nearest three points”, it is shown more specifically in FIG. 46.

The arrangement of X-ray detector channels in the multi-row X-ray detector 24 or two-dimensional X-ray area detector 24 of this embodiment 1 is as shown in FIG. 46. Marks “●” denote the central position (the position of the center of gravity) of each X-ray detector channel.

When data at the point “▪” are to be obtained by weighted addition processing, since the point “▪” is located in ΔEFG, it can be figured out by weighted addition processing of data at the three points including point E, point F and point G.

When data at the point “▴” are to be similarly obtained by weighted addition processing, since the point “▴” is located in (FGH, it can be figured out by weighted addition processing of data at the three points including point F, point G and point H.

Thus, points contained within the triangle in FIG. 46(a) can be figured out by subjecting the data at the three apexes of the triangle to weighted addition processing.

Further, where points are contained in the quadrangle ABCD in FIG. 46(a), as shown in FIG. 46(b), three points can be selected and determined in the following way. When “x” is in the lower left part of the quadrangle ABCD as shown in FIG. 46(c), point A, point C and point D of ΔACD are selected as shown in FIG. 46(d), for three-point interpolation, and when “x” is in the lower right part of the quadrangle ABCD as shown in FIG. 46(e), point B, point C and point D of ΔBCD are selected as shown in FIG. 46(f), for three-point interpolation.

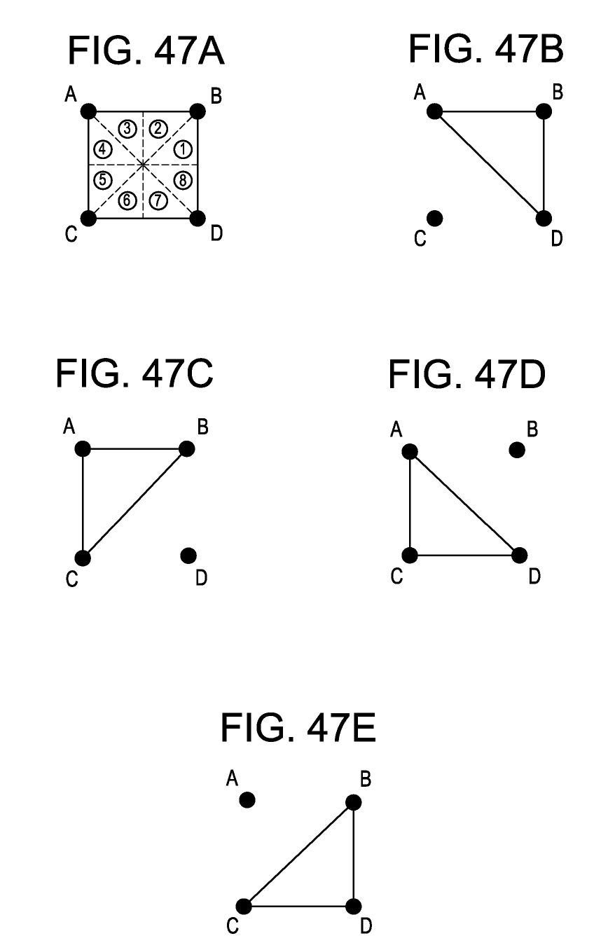

Further, details of this classification into different cases are shown in FIG. 47.

As shown in FIG. 47(a), where the quadrangle ABCD is divided into eight quadrants 1 through 8, point A, point B and point D of ΔABD as shown in FIG. 47 (b)are selected in the case of quadrants 1 and 2, point A, point B and point C of ΔABC as shown in FIG. 47(c) are selected in the case of quadrants 2 and 4, point A, point C and point D of ΔACD as shown in FIG. 47(d)are selected in the case of quadrants 5 and 6, and point B, point C and point D of ABCD as shown in FIG. 47(e)are selected in the case of quadrants 7 and 8.

Incidentally, the above-described idea of three-point weighted addition can be similarly applied to interpolation processing.

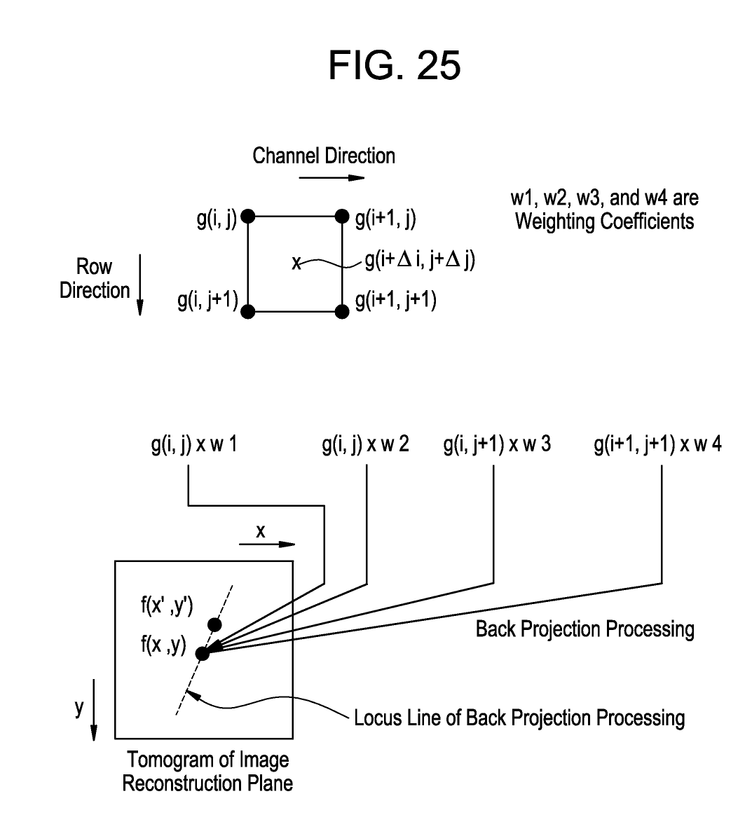

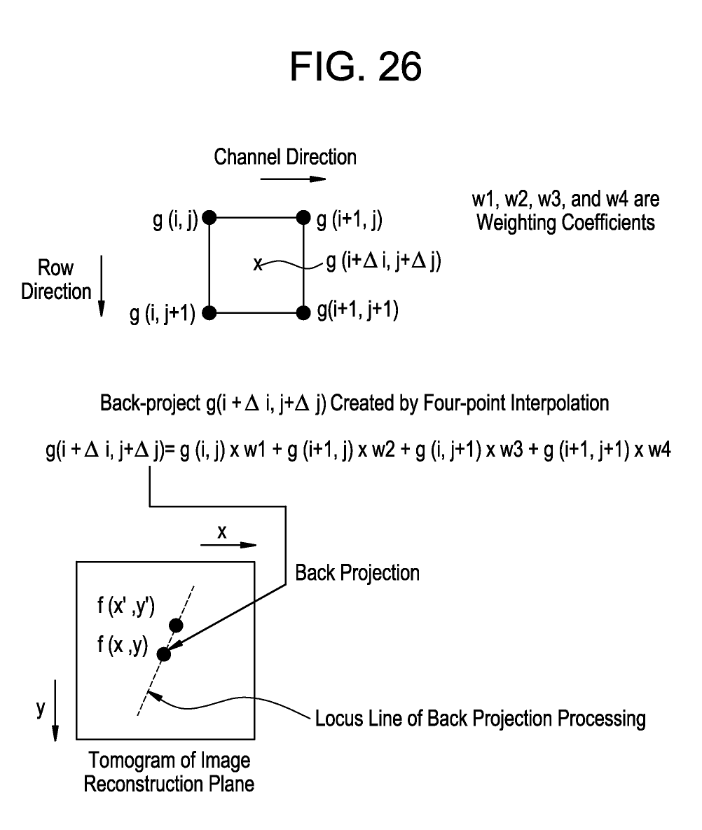

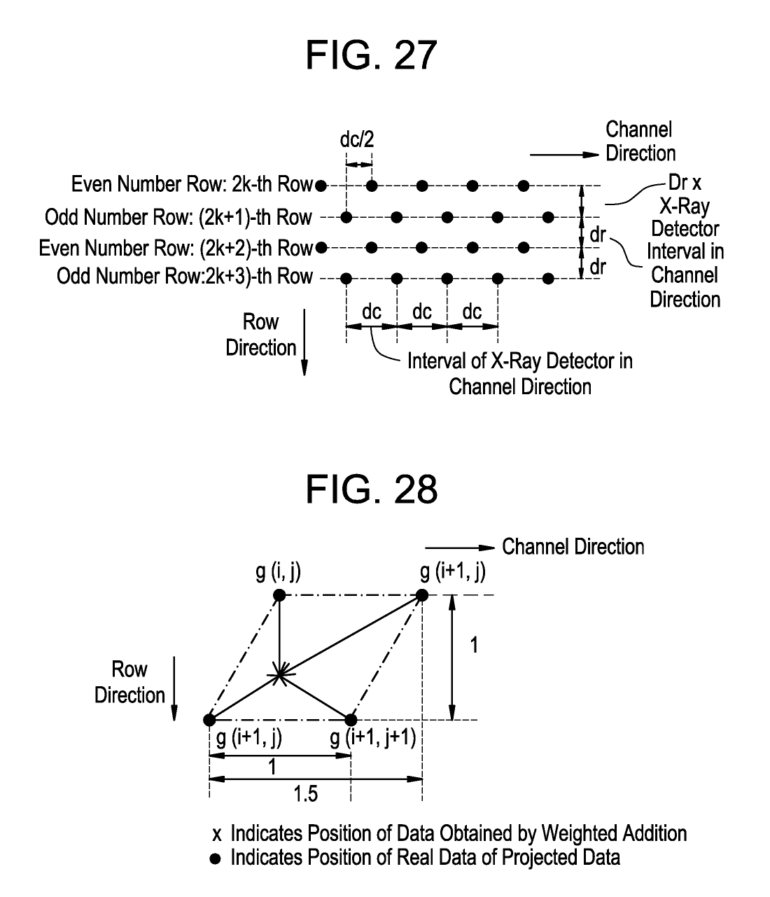

Application of weighted addition processing to interpolation processing will be described with reference to FIG. 25 and FIG. 26.

First with reference to FIG. 25, detailed differences between weighted addition processing and interpolation processing will be described. Incidentally, the description here will refer in particular to a case in which data are extracted from X-ray projection data at the time of three-dimensional image reconstruction and three-dimensional back projection is processed on a tomogram on the image reconstruction plane.

FIG. 25 shows a case of back projection processing by four-point weighted addition. Now, it is supposed that the point g(i+Δi, j+Δj) on the X-ray projection data to be back-projected is figured out and it is back-projected onto a tomogram on the image reconstruction plane. The real data of the X-ray projection data in the vicinity of the point g(i+Δi, j +Δj) being supposed to be g(i, j), g(i+1, j), g(i, j +1) and g(i+1, j+1), if the weighting coefficients w1, w2, w3 and w4 are so determined as to let the following equation hold:

[Mathematical Expression 18]
g(i+Δi, j+Δj)=g(i, j)×w1+g(i+1, j)×w2+g(i, j+1)×w3+g(i+1, j+1)×w4
instead of figuring out the point g(i+Δi, j+Δj) from the foregoing equation, the products of X-ray projection data multiplied by the four-point weighting coefficients on the X-ray projection data matching the pixels of the tomogram on the image reconstruction plane while scanning the image reconstruction plane:
w1×g(i, j)
w2×g(i+1, j)
w3×g(i, j+1)
w4×g(i+1, j+1)
are added to the pixels (x, y) of the tomogram on the image reconstruction plane.

On the other hand, in contrast to it, a case of back projection processing by four-point interpolation is shown in FIG. 26.

Now, it is supposed that the point g(i+Δi, j+Δj) on the X-ray projection data to be back-projected is figured out and it is back-projected onto a tomogram on the image reconstruction plane. The real data of the X-ray projection data in the vicinity of the point g(i+Δi, j+Δj) being supposed to be g(i, j), g(i+1, j), g(i, j+1) and g(i+1, j+1), if the weighting coefficients w1, w2, w3 and w4 are so determined as to let the following equation hold:

[Mathematical Expression 19]
g(+Δi, j+Δj)=g(i, j)×w1+g(i+1, j)×w2+g(i, j+1)×w3+g(i+1, j+1)×w4

the point (i +Δi, j +Δj) is figured out from the foregoing equation. While matching X-ray projection data with the tomogram pixel data along with the scanning of the image reconstruction plane, interpolation coefficients w1, w2, w3 and w4, which are figured out, are added to the pixels f(x, y) of the tomogram on the image reconstruction plane in search of g(i+Δi, j+Δj) having undergone data extraction by the four-point interpolation described above.

In this way, when three-dimensional back projection is to be carried out to the pixels f(x, y) of the tomogram of the image reconstruction plane, whether in weighted addition processing or in interpolation processing, eventually addition of the under-mentioned point g(i+Δi, j+Δj)to f(x, y) takes place, so that there seems to be no mathematical difference between them.

[Mathematical Expression 20]
g(i+Δi, j+Δj)=g(i, j)×w1+g(i+1, j)×w2+g(i, j+1)×w3+g(i+1, j+1)×w4

However, in back projection processing or three-dimensional back projection processing, g(i+Δi, j+Δj) is added to the tomogram of the back projection image reconstruction plane on the back projection processing locus line as shown in FIG. 25 and FIG. 26. The tomogram actually is composed of points “●” of the lattice coordinate system (Cartesian system) as shown in FIG. 34.

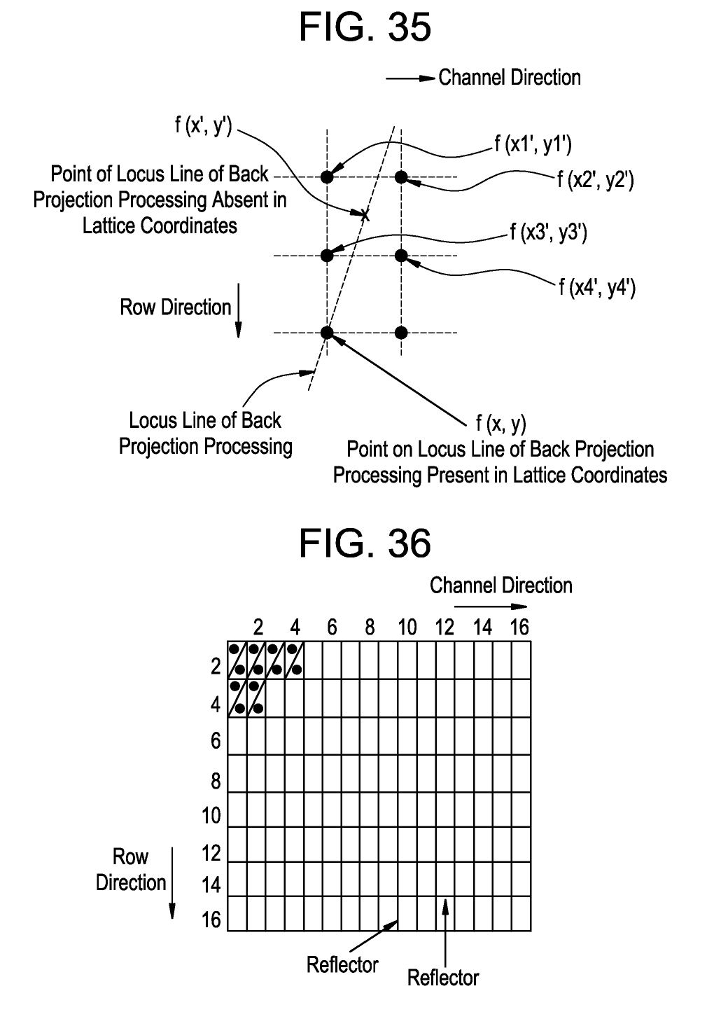

In this case, the back projection processing locus line does not necessarily pass only the lattice points of this lattice coordinate system. It is considered a case in which, for instance, addition of back projection processing of g(i +Δi, j+Δj) to a pixel f(x′, y′) in the vicinity of f(x, y) on the same back projection processing locus line as the pixel f(x, y) on the tomogram is to be performed. Supposing that f(x′, y′) is not on a lattice coordinate point and lattice coordinate points near f(x′, y′) are f(x1′, y1′), f(x2′, y2′), f(x3′, y3′) and f(x4′, y4′) as shown in FIG. 35, in weighted addition processing, X-ray projection data g(i+Δi1, j+Δj1) matching the pixel f(x1′, y1′) on the tomogram are figured out as stated below and added to f(x1′, y1′).

[Mathematical Expression 21]

g(+Δi1),j+Δj1)=g(i, j)×w11+g(i+1, j)×w21+g(i, j+1)×w31+g(i+1, j+1)×w4

X-ray projection data g(i+Δi2, j+Δj2) matching the pixel f(x2′, y2′) on the tomogram are figured out in the following way, and added to f(x2′, y2′).

[Mathematical Expression 22]
g(i +Δi2), j+Δj2)=g(i, j)×w12+g(i+1, j) ×w22+g(i, j+1)×w32+g(i1, j1)×w42

X-ray projection data g(i+Δi3, j+Δj3) matching the pixel f(x3′, y3′) on the tomogram are figured out in the following way, and added to f(x3′, y3′).

[Mathematical Expression 23]
g(i+Δi3), j+Δj3)=g(i, j)×w13+g(i+1, j)×w23+g(i, j+1)×w33+g(i+1, j+1)×w43

X-ray projection data g(i+Δi4, j+Δj4) matching the pixel f(x4′, y4′) on the tomogram are figured out in the following way, and added to f(x4′, y4′).

[Mathematical Expression 24]
g(i+Δi4), j+j4)=g(i, j)×w14+g(i+1, j)×w24+g(i, j+1)×w34+g(i+1, j+1)×w44

Weighting coefficients w1x, w2x, w3x and w4x are newly figured out for the respective lattice coordinate points f(x1′, y1′), f(x2′, y2′), f(x3′, y3′) and f(x4′, y4′) in the vicinities of f(x′, y′), and subjected to weighted addition processing.

Further, where the point on the X-ray projection data matching the pixel f(x, y) the tomogram of the image reconstruction plane in interpolation processing is represented by g(i+Δi, j+Δj) and g(i+Δi, j+Δj) obtained by interpolation processing is represented by g1(k, 1), data on the nearby X-ray projection data are as follows.

In this case, the pixel f(x′, y′) in the vicinities of f(x, y) on the same back projection processing locus line as the pixel f(x, y) on the tomogram is as follows.

[Mathematical Expression 25]
f(x′, y′)=g1(k, l)×wa1+g1(k+1, l) ×wa2+g1(k, l+1)×wa3+g1(k+1, l+1) ×wa4

In this way, f(x′, y′) can be obtained from the data deriving from interpolation processing.

Thus, when three-dimensional back projection is carried out by using weighted addition processing, a tomogram can be obtained by three-dimensional back projection processing without letting the resolution of X-ray projection data deteriorate.

By contrast, when interpolation processing is used, the resolution of the tomogram obtained by three-dimensional back projection processing will deteriorate unless the resolution of the X-ray projection data converted by interpolation processing is sufficient. Conversely, even if interpolation processing is used, if the resolution of the converted X-ray projection data is sufficient, the resolution of the tomogram obtained by three-dimensional back projection processing will not deteriorate.

As described above, data to be back-projected were extracted by using three-point weighted addition processing or three-point interpolation processing, followed by three-dimensional back projection processing. However, even if data to be back-projected are extracted by four-point weighted addition processing or four-point interpolation processing and three-dimensional back projection is processed after that as shown in FIG. 28, the resolution in the channel direction may somewhat deteriorate, but a tomogram having a higher resolution that is shown in FIG. 21 can be obtained.

### Embodiment 2

Embodiment 2 shown in FIG. 36 is a version of Embodiment 1 in which the peripheral parts of the X-ray detector module are made easier to fabricate.

In Embodiment 2, a hound's tooth check structure substantially similar to that in Embodiment 1 is used.

In Embodiment 2 as well, pre-treatments, reconstruction function convolution and so forth are processed similarly to pre-treatments at step S2, beam hardening correction at step S3 and z-filter convolution processing at step S4, reconstruction function convolution at step S5 and post-treatments at step S7.

In three-dimensional back projection processing at step S6, by similarly using three-point weighted addition processing of Embodiment 1, data extraction can be accomplished without blurring projection data, and image reconstruction can be achieved without deteriorating the spatial resolution of the tomogram obtained by three-dimensional back projection processing.

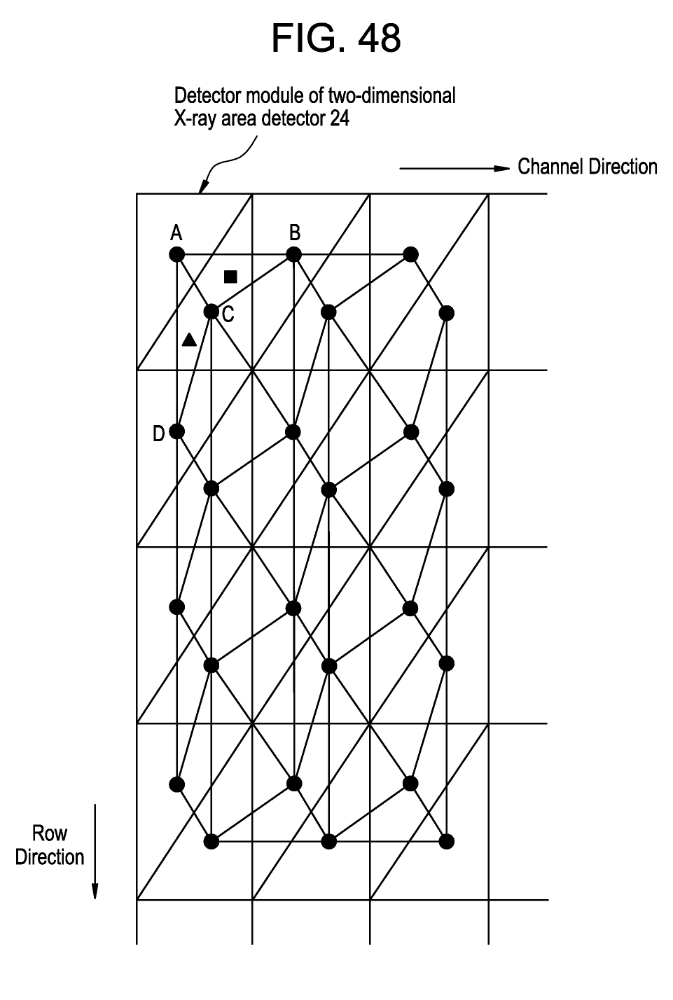

The way of selecting the three points in the three-point weighted addition processing or three-point interpolation processing in this Embodiment 2 basically is “to select the nearest three points”. FIG. 48 shows the way of selecting the three points in the three-point weighted addition processing or three-point interpolation processing in Embodiment 2.

The arrangement of X-ray detector channels in the multi-row X-ray detector 24 or two-dimensional X-ray area detector 24 in Embodiment 2 is as shown in FIG. 48. Marks “●” denote the central position (the position of the center of gravity) of each X-ray detector channel.

When data at the point “▪” are to be obtained by weighted addition processing, since the point “▪” is located in ΔABC, it can be figured out by weighted addition processing of data at the three points including point A, point B and point C.

When data at the point “▴” are to be similarly obtained by weighted addition processing, since the point “▴” is located in ΔACD, it can be figured out by weighted addition processing of data at the three points including point A, point C and point D.

FIG. 48, unlike FIG. 46 illustrating the way of three points in Embodiment 1, there is no case of quadrangle, but every case is configured in a triangle. For this reason, the three points to be selected are always determined uniquely.

### Embodiment 3

In contrast to X-ray projection data obtained from X-ray detectors by the X-ray detector module shown in FIG. 23 or FIG. 36,

X-ray projection data having gone through pre-treatments at step S2 of FIG. 5, X-ray projection data having gone through beam hardening correct at step S3 of FIG. 5 or X-ray projection data having gone through z-filter convolution processing at step S4 of FIG. 5 being represented by D(view, j, i), interleaving by alternately inserting X-ray detector data in the channel direction into the j-th row X-ray detector data D(view, j, i) of X-ray detectors and the (j+1)-th row X-ray detector data D(view, j+1, i) of X-ray detectors can give new k-th row 1-th channel X-ray detector data D(view, k, 1).

Provided that 1≦1≦2·CH, 1≦k≦ROW/2.

For instance, D1(view, 1, 1)=(D(view, 1, 1), D(view, 2, 1),
D(view, 1, 2), D(view, 2, 2),
D(view, 1, 3), D(view, 2, 3),
... ...
D(view, 1, CH), D(view, 2, CH).

Namely, D1(view, 2j +1)=D(view, j, int(1/2)),
D1(view, 2j, 1)=D(view, j, int(1/2)).

This serves to enhance the resolution of X-ray projection data in the channel direction, thereby enabling the spatial resolution of the tomogram to be enhanced.

Where the distance between the j-th row and the (j+1)-th row is negligible relative to the slice thickness, even if more or less artifact due to a lag between the j-th row and the (j+1)-th row is generated, the foregoing method is effective where good performance of the tomogram is desired in terms of spatial resolution.

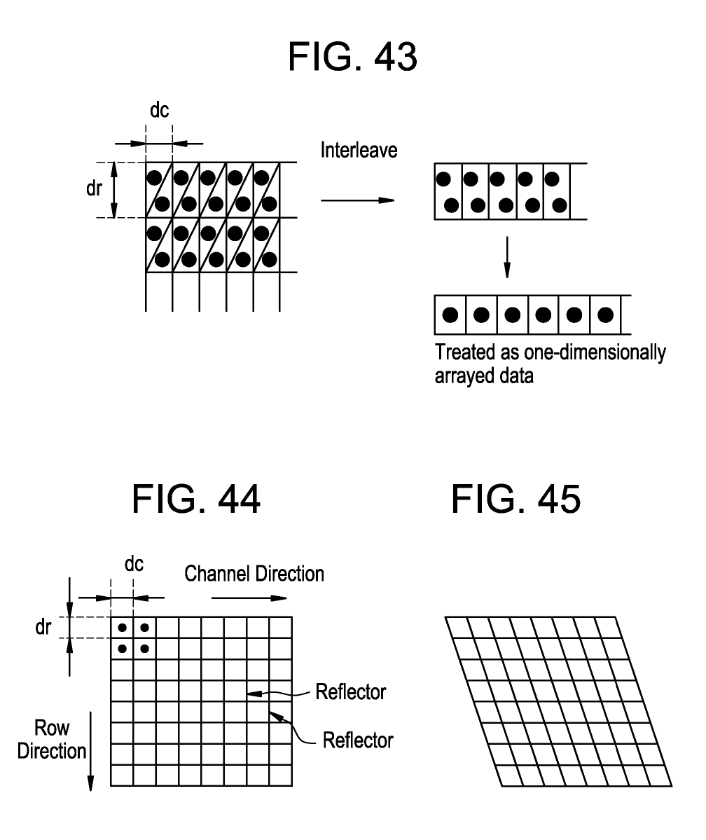

The X-ray projection data then interleaved can be treated as if they were one-dimensionally arrayed data as shown in FIG. 43. Especially where the slice thickness is sufficiently great relative to the row width dr, when X-ray projection data equivalent to that slice thickness are to be added in the row direction (z direction), if that slice thickness is great enough to make the row width dr negligible, such approximation will adequately hold.

Incidentally, it is also acceptable to extract data, after subjecting the X-ray projection data then interleaved to weighted addition or interpolation in the channel direction by two-point weighted addition or two-point interpolation, and to perform three-dimensional back projection processing.

It also acceptable to perform nearest neighbor processing which bring about the “nearest data” instead of two-point weighted addition or two-point interpolation, extract data and perform three-dimensional back projection processing.

### Embodiment 4

To compare three-point weighted addition processing and three-point interpolation processing with four-point weighted addition processing and four-point interpolation processing, there are found the following general differences.

(1) Three-point weighted addition processing and three-point interpolation processing: Poor in S/N ratio, but good in resolution.

(2) Four-point weighted addition processing and four-point interpolation processing: Good in S/N ratio, but poor in resolution.

The difference in S/N ratio is due to the difference in the number of data used in weighted addition processing or interpolation processing; generally, the greater the number of data, the higher the S/N ratio and the lower the image noise.

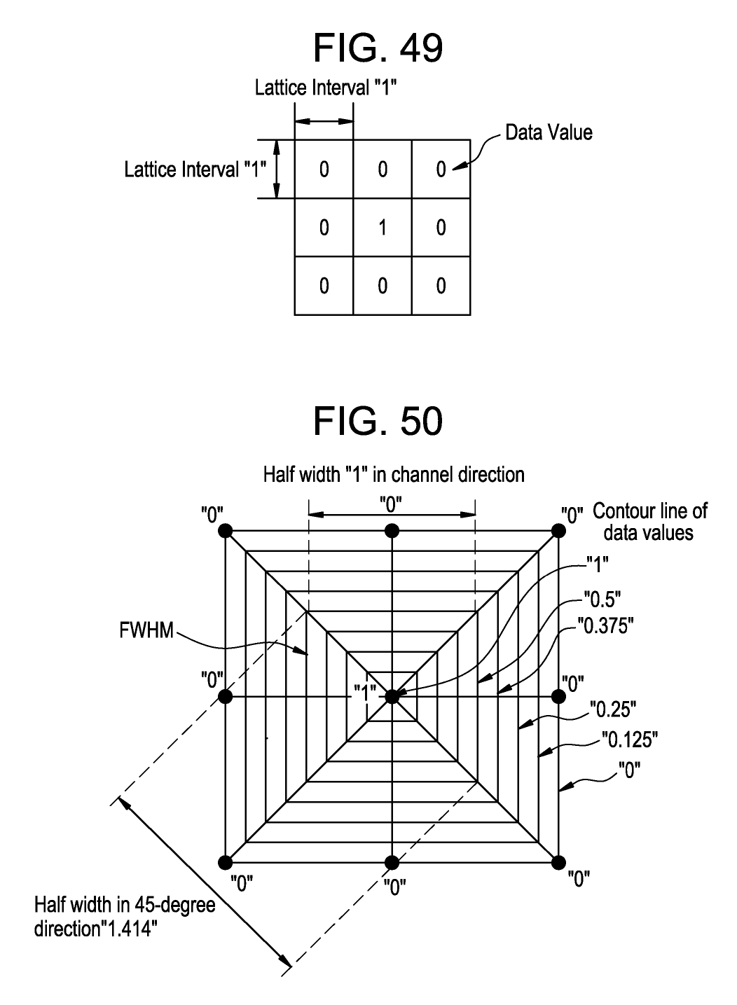

Facts about the resolution is shown in FIG. 49.

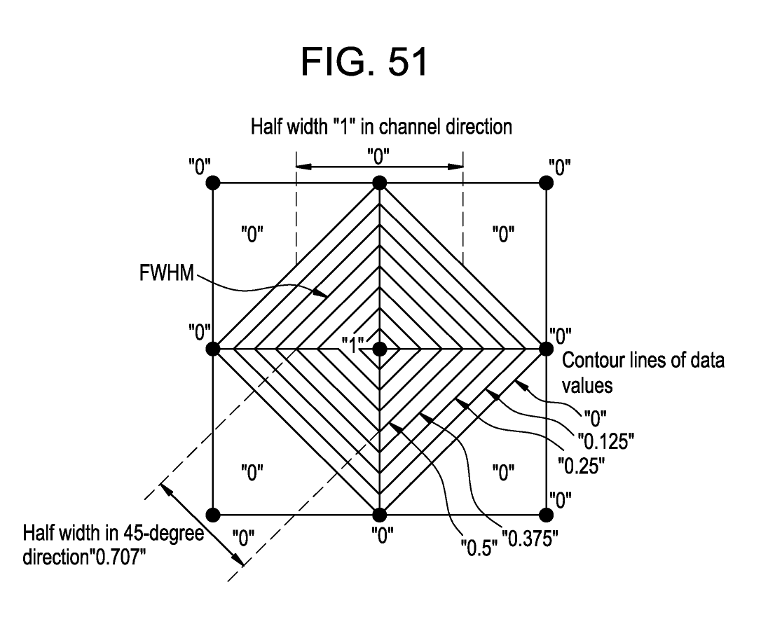

FIG. 49 shows data of some of the X-ray detector channels in the multi-row X-ray detector 24 or two-dimensional X-ray area 24. Herein, for the sake of ease of understanding, X-ray projection data indicating a high-frequency variation in which a datum of “1” is found on only one channel among “0” data of 3×3 channels. It is now considered a case in which, the interval of one lattice unit shown in FIG. 50 being supposed to be “1”, X-ray projection data are subjected to data extraction at fine intervals in four-point weighted addition processing or four-point interpolation processing. Referring to FIG. 50, when data are extracted at 0.125 intervals, in the four-point weighted addition processing or four-point interpolation processing shown in FIG. 50, while the half width FWHM (Full Width Half Maximum) is “1” in the horizontal direction and “1.414” in a 45-degree slanted direction relative to intervals of “1”, and in the three-point weighted addition processing or three-point interpolation processing shown in FIG. 51, the half width FWHM is “1” in the horizontal direction and “0.707” in a 45-degree slanted direction.

Thus it is seen that the resolution is higher in three-point weighted addition processing or three-point interpolation processing.

In Embodiment 4, pre-treatments at step S2, beam hardening correction at step S3 and z-filter convolution processing at step S4 shown in FIG. 5 are accomplished in the same was as in Embodiment 1. However, in the fan-to-parallel conversion of converting the X-ray projection data of the final fan beam into X-ray projection data of a parallel beam in the z-filter convolution processing at step S4, three-point weighted addition processing or three-point interpolation processing may as well be used.

If three-point weighted addition processing or three-point interpolation processing is used in the three-dimensional back projection processing at step S6 after the processing until the reconstruction function convolution at step S5 is accomplished in the same way as in Embodiment 1, the resolution of the tomogram may prove higher than when four-point weighted addition processing or four-point interpolation processing is used.

In this way, the resolution of the tomogram can be improved by three-point weighted addition processing or three-point.

In the X-ray CT apparatus 100 described so far, the X-ray CT apparatus or the X-ray CT imaging method according to the present invention can realize by a simple method achievement of higher X-ray detector resolution for multi-row X-ray detectors or two-dimensional X-ray area detectors of matrix structure, and to realize enhancement of the resolution of tomograms by an X-ray CT apparatus using such X-ray detectors by conventional scanning (axial scanning), cine-scanning, helical scanning or variable-pitch helical scanning.

Incidentally, the image reconstruction method in this embodiment may be the usual three-dimensional image reconstruction method according to the already known Feldkamp method. It may even be some other three-dimensional image reconstructing method. Alternatively, it may be two-dimensional image reconstruction.

Also, a uniform slice thickness from row to row and picture quality in terms of artifact and noise are achieved in this embodiment by convoluting row-direction (z-direction) filters differing in coefficient from row to row thereby to adjust fluctuations in picture quality, and various z-direction filter coefficients are conceivable for this purpose. Any of which can give a similar effect.

Although this embodiment has been described under the assumption of using the X-ray CT apparatus for medical purposes, it can as well be utilized as an X-ray CT apparatus for industrial purposes or an X-ray CT-PET apparatus or an X-ray CT-SPECT apparatus in combination with some other apparatus.

Although this embodiment uses weighted addition or interpolation in three-point weighted addition or three-point interpolation by linear approximation, higher order weighted addition or interpolation, such as the second order or the third order, may as well be used.

Although the X-ray detector module is supposed to be rectangular as shown in FIG. 44, it may as well be a parallelogrammatic X-ray detector module as shown in FIG. 45. In this case, since the X-ray detector channel in an end part will have the same shape as the X-ray detector channel in the central part, the problem with the X-ray detector channel in an end part as shown in FIG. 23 will not arise.

1. An X-ray CT apparatus comprising:
X-ray data acquisition device for acquiring projection data of an X-ray passed through a subject positioned between an X-ray generator and an X-ray detector which are opposite to each other;
image reconstructing device for performing image reconstruction from the projection data acquired from that X-ray data acquisition device;
image display device for displaying a tomographic image obtained by said image reconstructing device; and
imaging condition setting device for setting various image acquisition parameters for acquisition of a tomographic image,
wherein said X-ray detector includes a detector of which the X-ray detector module is divided into X-ray detector channels by parallel lines in three or more directions.

2. The X-ray CT apparatus according to claim 1, wherein:
said X-ray detector includes a multi-row detector.

3. The X-ray CT apparatus according to claim 1, wherein:
said X-ray detector includes a two-dimensional X-ray area detector.

4. The X-ray CT apparatus according to claim 1, wherein:
said X-ray detector channel has a triangular shape.

5. An X-ray CT apparatus comprising:
X-ray data acquisition device for acquiring projection data of an X-ray passed through a subject positioned between an X-ray generator and an X-ray detector which are opposite to each other;
image reconstructing device for performing image reconstruction from the projection data acquired from that X-ray data acquisition device;
image display device for displaying a tomographic image obtained by said image reconstructing device; and
imaging condition setting device for setting various image acquisition parameters for acquisition of a tomographic image,
wherein said image reconstructing device includes three-point weighted addition processing or three-point interpolation processing.

6. The X-ray CT apparatus according to claim 1, wherein:
said image reconstructing device includes three-point weighted addition processing or three-point interpolation processing.

7. The X-ray CT apparatus according to claim 1, wherein:
said image reconstructing device includes four-point weighted addition processing or four-point interpolation processing.

8. The X-ray CT apparatus according to claim 1, wherein:
said image reconstructing device includes two-point weighted addition processing or two-point interpolation processing.

9. The X-ray CT apparatus according to claim 1, wherein:
said image reconstructing device includes nearest neighbor processing.

10. The X-ray CT apparatus according to claim 1, wherein:
said image reconstructing device includes three-dimensional image reconstruction processing.

11. The X-ray CT apparatus according to claim 10, wherein:
said image reconstructing device includes units for performing image reconstruction of a tomogram of any desired slice thickness in any z-direction coordinate position when conventional scanning (axial scanning) or cine-scanning is performed.

12. The X-ray CT apparatus according to claim 10, wherein:
said image reconstructing device includes units for performing image reconstruction of a tomogram of any desired slice thickness in any z-direction coordinate position when helical scanning or variable-pitch helical scanning.

13. The X-ray CT apparatus according to claim 11, wherein:
said image reconstructing device includes units for performing image reconstruction includes device for alternately rearranges and interleaves X-ray projection data on adjoining rows, reconstructs high-resolution X-ray projection data and performs image reconstruction of the X-ray projection data.

14. The X-ray CT apparatus according to claim 12, wherein:
said image reconstructing device includes units for performing image reconstruction includes device for alternately rearranges and interleaves X-ray projection data on adjoining rows, reconstructs high-resolution X-ray projection data and performs image reconstruction of the X-ray projection data.

15. The X-ray CT apparatus according to claim 13, wherein:
said image reconstructing device includes units for performing image reconstruction includes units for alternately rearranges and interleaves X-ray projection data on adjoining rows in the case of a high-frequency reconstruction function.

16. The X-ray CT apparatus according to claim 14, wherein:
said image reconstructing device includes units for performing image reconstruction includes units for alternately rearranges and interleaves X-ray projection data on adjoining rows in the case of a high-frequency reconstruction function.
